(19) United States
(12) Patent Application Publication
(10) Pub. No.: US 2021/0118571 A1
(43) Pub. Date: Apr. 22, 2021

(54) SYSTEM AND METHOD FOR DELIVERING POLYGENIC-BASED PREDICTIONS OF COMPLEX TRAITS AND RISKS

(71) Applicant: Board of Trustees of Michigan State University, East Lansing, MI (US)

(72) Inventors: Stephen D. H. Hsu, East Lansing, MI (US); Laurent C. A. M. Tellier, East Lansing, MI (US); Soke Yuen Yong, East Lansing, MI (US); Timothy G. Raben, East Lansing, MI (US); Louis Lello, East Lansing, MI (US)

(21) Appl. No.: 17/073,377

(22) Filed: Oct. 18, 2020

Related U.S. Application Data
(60) Provisional application No. 62/923,097, filed on Oct. 18, 2019.

(51) Int. Cl. G16H 50/30; G16B 20/20; G16B 30/00; G16B 40/00; G06N 7/00

(52) U.S. Cl. CPC G16H 50/30; G16B 20/20; G06N 7/005; G16B 40/00; G16B 30/00

(57) ABSTRACT

A process for providing a polygenic disease risk score for a patient calculated based on genomic data is provided by the disclosure. The polygenic disease risk score can be calculated further based on age and sex of the patient.

### CROSS-REFERENCE TO RELATED APPLICATIONS

This application claims priority to U.S. Provisional Application No. 62/923,097, filed on Oct. 18, 2019, which is herein incorporated by reference in full.

### STATEMENT REGARDING FEDERALLY SPONSORED RESEARCH

None.

### BACKGROUND

Many disease conditions and other important phenotypic characteristics are known to be significantly heritable. For example, it has long been recognized that traits like plant size or hardiness, or phenotypic characteristics like human eye color are significantly heritable, as are risks of certain genetic-based diseases. Various approaches have been taken in the past to generate predictions of specific phenotypes of various living organisms, or to attempt to predict disease conditions in plants and animals. These approaches have generally used either heuristic approaches (e.g., based on plant breeding or identification of specific gene mutations that are found to cause a certain protein activity) or some basic algorithmic methods from genetic data. The approaches have largely entailed predictions that isolate only a single phenotype or small set of phenotypes. Moreover, even when past attempts have utilized genomic data to generate predictions, they have tended to focus on phenotypes or disease conditions that have a less complex genomic indicator. For example, some work has focused on identifying a specific gene mutation as being correlated with or causing a specific disease.

However, robust techniques for prediction of more complex human traits and disease risks are currently lacking. Moreover, existing techniques are too narrowly focused to serve as a broad screening technique for multiple disease conditions and characteristics. Earlier studies have shown some narrow success on specific complex human disease risk using small datasets and a variety of methods. For example, early work in this direction can has included approaches using dense marker data sets, genome-wide allele significance from association studies in additive models, regression analysis, and accounting for linkage disequilibrium. But none of these approaches provides an accurate, consistent, single approach for prediction of a large set of complex traits from a single genotypic dataset.

A need exists for a consistent and accurate method that utilizes the entire genome to predict complex human phenotypes and to screen individuals for a broad range of disease risks. Using such a method, inexpensive genotyping (e.g., an array genotype which directly measures a million or more single-nucleotide polymorphisms (SNPs), and allows imputation of millions more) could be leveraged to identify individuals who are outliers in risk score, and hence are candidates for additional diagnostic testing, close observation, or preventative.

### SUMMARY

The present disclosure provides systems and methods for polygenic disease risk score predictor models.

In one aspect, the disclosure provides a method for generating a complex genomic predictor model comprising: obtaining a set of genomic data; pre-processing the genomic data set for at least one characteristic of interest; computing a set of additive effects that minimize an objective function for the characteristic of interest in the pre-processed genomic data set; and determining a polygenic risk score predictor model for the at least one characteristic of interest.

In another aspect, the disclosure provides a method for providing a polygenic risk score, comprising: obtaining genotype data associated with an individual; pre-processing the genotype data; inputting the genotype data to a polygenic risk score predictor model, wherein the predictor model was developed through a penalized, modified LASSO regression applied to determine a set of predictor SNPs from a training genomic data set; obtaining at least one risk score of a trait of interest for the individual from the predictor model; and outputting a report based on a risk score for the trait of interest for the individual, according to user output preferences.

In yet another aspect, the disclosure provides a system for providing polygenic risk scores, the system comprising: a processor; at least one memory associated with the processor, the memory comprising: a database of training records, each record comprising genomic information of an individual and at least one characteristic of the individual; a set of instructions which, when executed by the processor, cause the processor to: receive genotype information for a user; pre-process the genotype information to determine whether a threshold of SNP information is present; provide the genotype information to a polygenic risk score predictor model; output a report for the user based upon the result of the polygenic risk score predictor model; and update the database of training records with the genotype information for the user, based on user consent.

The foregoing and other aspects and advantages of the invention will appear from the following description. In the description, reference is made to the accompanying drawings that form a part hereof, and in which there is shown by way of illustration one or more preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph of an exemplary receiver operating characteristic curve.

FIG. 2A is a graph of area under curve (AUC) for a hypertension predictor model trained using a UK Biobank dataset.

FIG. 2B is a graph of area under curve (AUC) for a hypertension predictor model trained using an eMERGE dataset.

FIG. 3A is a graph of a distribution of polygenic score (PGS), cases and controls for Hypertension in the eMERGE dataset using single-nucleotide polymorphisms (SNPs) alone.

FIG. 3B is a graph of distribution of PGS, cases and controls for Hypertension in the eMERGE dataset using sex and age as regressors.

FIG. 4A is a graph of distribution of PGS, cases and controls for Resistant Hypertension in the eMERGE dataset using SNPs alone.

FIG. 4B is a graph of distribution of PGS, cases and controls for Resistant Hypertension in the eMERGE dataset using sex and age as regressors.

FIG. 5A is a graph of distribution of PGS, cases and controls for Hypothyroidism in the eMERGE dataset using SNPs alone.

FIG. 5B is a graph of distribution of PGS, cases and controls for Hypothyroidism in the eMERGE dataset using sex and age as regressors.

FIG. 6A is a graph of distribution of PGS, cases and controls for type 2 diabetes in the eMERGE dataset using SNPs alone.

FIG. 6B is a graph of distribution of PGS, cases and controls for type 2 diabetes in the eMERGE dataset using sex and age as regressors.

FIG. 7A is a graph of Odds ratio between upper percentile in PGS and total population prevalence in eMERGE for Hypothyroidism using SNPs alone.

FIG. 7B is a graph of Odds ratio between upper percentile in PGS and total population prevalence in eMERGE for Hypothyroidism using SNPs with sex and age as covariates.

FIG. 8A is a graph of Odds ratio between upper percentile in PGS and total population prevalence in eMERGE for Hypertension using SNPs alone.

FIG. 8B is a graph of Odds ratio between upper percentile in PGS and total population prevalence in eMERGE for Hypertension using SNPs with sex and age as covariates.

FIG. 9A is a graph of Odds ratio between upper percentile in PGS and total population prevalence in eMERGE for Resistant Hypertension using SNPs alone.

FIG. 9B is a graph of Odds ratio between upper percentile in PGS and total population prevalence in eMERGE for Resistant Hypertension using SNPs with sex and age as covariates.

FIG. 10A is a graph of Odds ratio between upper percentile in PGS and total population prevalence in eMERGE for Type 2 Diabetes using SNPs alone.

FIG. 10B is a graph of Odds ratio between upper percentile in PGS and total population prevalence in eMERGE for Type 2 Diabetes using SNPs with sex and age as covariates.

FIG. 11 is a graph of a graph of maximum AUC on eMERGE as a function of the number of cases in thousands included in training for type 2 diabetes, Hypothyroidism and Hypertension.

FIG. 12A is a graph of odds ratio as a function of PGS percentile for Asthma.

FIG. 12B is a graph of odds ratio as a function of PGS percentile for Atrial Fibrillation.

FIG. 13A is a graph of odds ratio as a function of PGS percentile for Basal Cell Carcinoma.

FIG. 13B is a graph of odds ratio as a function of PGS percentile for Breast cancer.

FIG. 14A is a graph of odds ratio as a function of PGS percentile for Gallstones.

FIG. 14B is a graph of odds ratio as a function of PGS percentile for Gluacoma.

FIG. 15A is a graph of odds ratio as a function of PGS percentile for Gout.

FIG. 15B is a graph of odds ratio as a function of PGS percentile for Heart Attack.

FIG. 16A is a graph of odds ratio as a function of PGS percentile for High Cholesterol.

FIG. 16B is a graph of odds ratio as a function of PGS percentile for Malignant Melanoma.

FIG. 17A is a graph of odds ratio as a function of PGS percentile for Type 1 Diabetes.

FIG. 17B is a graph of odds ratio as a function of PGS percentile for Testicular Cancer.

FIG. 18 is a graph of odds ratio as a function of PGS percentile (i.e. scores at that point or above) for Prostate Cancer.

FIG. 19A is a graph of the odds ratio as a function of AUC for z-scores above the 98^{th} percentile at various values of the ratio of cases to controls r.

FIG. 19B is a graph of the odds ratio as a function of AUC for case to control ratio r=0.1 at various z-score percentiles.

FIG. 20 is an exemplary network block diagram demonstrating an example embodiment of a system for generating and providing disease risk scores to users.

FIG. 21 is an exemplary process for providing a polygenic disease risk score based on genomic data.

FIG. 22 is an exemplary process for training and updating a phenotypic predictor model.

FIG. 23 is an exemplary system for predicting one or more genomic risk scores of a patient.

FIG. 24 is an exemplary system for providing previously generated genotype and phenotype data to a trained predictor model.

### DETAILED DESCRIPTION

Various systems and methods are disclosed herein for overcoming the disadvantages of the prior art.

As mentioned above, many important disease conditions are known to be significantly heritable. The significant heritability of common disease conditions implies that at least some of the variance in risk is due to genetic effects. With enough training data, the various statistical and machine learning techniques disclosed herein can enable the construction of polygenic predictors of risk of certain diseases, or likelihood of certain traits. An algorithm as disclosed herein, when implemented within a system allowing access to enough examples to train on, can eventually identify individuals, based on genotype alone, who are at unusually high risk for certain conditions. This has obvious clinical applications: scarce resources for prevention and diagnosis can be more efficiently allocated if high risk individuals can be identified while still negative for the disease condition. This identification can occur early in life, or even before birth.

For the several experiments described herein, UK Biobank data was used to construct predictors for a number of conditions. Out of sample testing was conducted using eMERGE data (collected from the US population) and Ancestry Out of Sample (AOS) testing using UK ethnic subgroups distinct from the training population. The results suggest that the generated polygenic scores indeed predict complex disease risk—there is very strong agreement in performance between the training and out of sample testing populations. Furthermore, in both the training and test populations the distribution of PGS is approximately Gaussian, with cases having on average higher scores. For all disease conditions studied, a simple model of displaced Gaussian distributions predicts empirically observed odds ratios (i.e., individual risk in test population) was verified as a function of PGS. This is strong evidence that the polygenic score itself, generated for each disease condition using machine learning, is indeed capturing a nontrivial component of genetic risk.

By varying the amount of case data used in training, the rate of improvement of polygenic predictors was estimated with sample size. Sample datasets of sufficient sizes are readily, and with the will result in predictors of significant clinical utility. Additionally, extending this analysis to exome and whole genome data will also improve prediction. The use of genomics in Precision Medicine has a bright future, which is just beginning. Thus, there is a strong case for making inexpensive genotyping Standard of Care in health systems across the world.

The inventors have thus developed methods and techniques which condition and prune datasets for optimal development of predictors through use of various unique machine learning and statistical methods. These predictors are then employed via new systems that can obtain specific genotyping data for a given individual (e.g., by a user uploading from a portal, direct link with a genotyping company's network, or a link with electronic medical records and similar healthcare software tools) to obtain a specific risk panel for that individual for a multitude (or a specified number) of heritable diseases, and deliver that risk estimate in an appropriate manner to healthcare professionals and other users.

As will be described herein, there are several different techniques that may be employed individually or in combination for data processing and predictor generation. While specific examples are described in detail, it should be understood that these techniques are adaptable and usable in a variety of combinations. Once a predictor is developed, it can then be employed in various system architectures to provide appropriate reports and recommendations to users.

The discussion below will begin with overview explanations of several discoveries, learnings from experimental analyses, and other insights and considerations which guided development of the methods and techniques herein. Then, example embodiments of particular methodologies for leveraging predictors (and systems to implement those methodologies) are described, in which genomic data can be modified and transformed, and then leveraged to generate robust predictor models capable of assessing risk of multiple disease conditions and/or heritable traits. The discussion will set forth details of various systems and methods for employing these predictor models to provide reports and screening to users.

### Overview of Data Processing and Predictor Generation Methods

For purposes of explanation of a first set of techniques and methods, an instance of developing a predictor model using a modified L1-penalized regression technique (e.g., a modified LASSO technique) will be described. In one study, this modified LASSO technique was used by the inventors to process case-control data from a dataset known as the UK Biobank (UKBB) and construct disease risk predictors. In other studies, the inventors demonstrated that a similar method can be used to predict quantitative traits such as height, bone density, and educational attainment. The height predictor that was generated captured almost all of the expected heritability for height and has a prediction error of roughly a few centimeters. Similar methods have also been employed by the inventors in work on other case-control datasets.

Collation and Pre-Processing of Datasets.

In a first example, the inventors conditioned and pruned the UKBB dataset. The inventors determined through their analyses that generating a predictor using homogenous data from a standpoint of genetic ancestry would yield more accurate results. Thus, only those records from the UKBB dataset representing genetically British individuals (as defined by UKBB using principal component analysis) were used for training of the predictors. However, validating a model created from such a homogenous dataset would benefit from use of data records that are not part of that dataset (otherwise known as “out of sample testing”). For out of sample testing, records from the “eMERGE” dataset (restricted to self-reported white Americans) was used in addition to self-reported white but nongenetically British individuals in UKBB. The specific eMERGE data set used here refers to data obtained from dbGaP, under accession phs000360.v3.p1 (https://www.ncbi.nlm.nih.gov/projects/gap/cgi-bin/study.cgi?study_id=phs000360.v3.p1). This latter testing method is referred to as Ancestry Out of Sample (AOS) testing: the individuals used are part of the UKBB dataset, but will not have been used in generating the predictor and differ in ancestry from the training population.

The UKBB and eMERGE datasets include genomic data as well as disease/diagnosis outcomes for hundreds of thousands of individuals. In some instances these datasets are age-limited (e.g., 40-69 years for the original UKBB dataset), though they are frequently linked to electronic medical record data which contains diagnosis codes (e.g., ICD9 or ICD10 codes), demographic information (e.g., age, gender, self-reported ethnicity), and time-series test results (e.g., blood pressure measurements, weight measurements, urine analyses, cholesterol counts, etc.). It should be understood, however, that one aspect of the techniques and systems disclosed herein is that they can be made adaptable to utilize any format of data records that includes genotype data and some trait or outcome information, to generate predictor models. For example, an initial dataset that correlates genotype data with robust patient diagnosis data such as the UKBB could be used to develop a predictor for a variety of cardiovascular diseases (based on the diagnosis codes included in the UKBB). Subsequently, new patient records acquired via the various methods described below could be added to the dataset or used to create a separate dataset for further training or validation that include genotype data and diagnosis outcomes, even if they lack the time series test results included in the UKBB. Or, alternatively, existing records could be updated as new diagnoses are made (e.g., a record that previously did not indicate a diagnosis of hypertension could be updated with that diagnosis if the corresponding patient is determined to have developed hypertension).

As will be further described below, one feature of the techniques and systems disclosed herein is a data pre-processing module. In one embodiment, this could be implemented as a software routine that receives one or more records of a dataset and performs a number of processes to condition the data to be more usable for purposes of generating, refining, or validating a predictor model.

The pre-processing module could first perform a quality-check on a given data record (or set of data records) to determine that they contained valid data (e.g., non-null fields, no corrupted data, and only like information within a given field of each record). The module could then either assess or confirm the types of data within each record. For example, the module could perform a genotype quality control and a phenotype quality control, to confirm whether the data records contain sufficient genomic data as well as which types of patient/demographic/phenotype data are included. For example, if a particular dataset did not include age, gender, diagnosis, or specific patient measurement information, the dataset may not be useful for purposes of training or refining a predictor for a disease risk or heritable trait that is correlated to that information (e.g., hypertension tends to occur in older individuals, so lacking age information would make it difficult to determine whether a given record lacked a hypertension diagnosis because the person was too young to have developed it yet). Based on the categories of phenotypic, demographic, and diagnosis and other information included in a dataset, it can be categorized as valid for purposes of use for predictors of specific traits or disease risks, as further described below.

Predictor Model Generation

In one embodiment, linear models of genetic predisposition can be constructed for a variety of disease conditions. While it would also be possible in other embodiments to utilize non-linear models to account for complex trait interaction, the inventors have found it helpful to leverage additive effects, which have been shown to account for much of the common single-nucleotide polymorphism (SNP) heritability for human phenotypes such as height, and in other plant and animal phenotypes. Thus, higher accuracy has been found to be achieved using linear models

The phenotype data included in a dataset to be used for generating a predictor model can be thought of as describing case-control status, in which “cases” are defined by whether the individual has been diagnosed for, or self-reports, the disease or trait condition of interest. The approach is built from an adaptation of compressed sensing techniques, based on which it has been shown that matrices of human genomes are good “sensing matrices” in the terminology of compressed sensing. That is, theorems resulting in performance guarantees and phase transition behavior of the L1 algorithms hold when human genome data are used. Furthermore, L1 penalization can efficiently capture expected common SNP heritability for complex traits (e.g., traits that are heritable based on multiple SNPs, rather than a single gene mutation). For example, human height, one of the most complex but highly heritable human traits, can be predicted using methods such as these. This ability to capture heritability for complex traits allows for the construction of clinically useful polygenic, multi-trait predictor systems, a fact that is not necessarily intuitive when simply analyzing a methodological comparison between different algorithms.

In one alterative, robust Bayesian Monte Carlo approaches that can account for a wide variety of model features like linkage disequilibrium and variable selection could be used in addition to or instead of the linear, L1 techniques mentioned above. However, for human complex traits, these methods may only produce a modest increase in predictive power at the cost of large computation times. Thus, they may be more useful for specific circumstances in which (1) large computational resources are available; (2) latency is acceptable; and (3) predictive accuracy is paramount (e.g., where a test for a specific disease would be highly invasive, such as a spinal tap or a biopsy of a sensitive organ). However, while the L1 methods are not explicitly Bayesian, posterior uncertainties can still be estimated in the predictor via repeated cross-validation.

Regardless of the specific method used to generate the predictor, a few decisions are made initially. First, a disease or trait of interest (or a set of such diseases/traits) is selected. Then, a system employing the techniques described herein will determine whether sufficient data exists to develop a predictor. For example, for highly rare diseases, only a few records in a given dataset might exist that contain that disease, meaning results might be overfitted or distorted. Likewise, in some embodiments, a priori knowledge of associations between a disease and non-genomic factors (like age, gender, weight, etc.) can be used to appropriately cull data. For example, where a disease is highly correlated with women, it may make sense to run the predictor generation techniques on a dataset of only women and on a dataset of both men and women. As another example, where a disease is highly correlated with age, only records within a dataset that have appropriate age information (e.g., over 50 years old) would be used.

Once it has been determined that sufficient data exists, for each disease condition of interest, a set of additive effects \vec{β}* (each component is the effect size for a specific SNP) is computed that minimizes the LASSO objective function as set forth in Equation 2.1:

O_{λ}(\vec{β})=½∥\vec{y}−X\vec{β}∥²+nλ∥\vec{β}∥₁; \vec{β}*= [symbol] O_{λ}(\vec{y},X;\vec{β}), (2.1)

where p is the number of regressands, n is the number of samples, ∥ . . . ∥ means L₂ norm (square root of sum of squares), ∥ . . . ∥₁ is the L₁ norm (sum of absolute values) and the term Ink is a penalization which enforces sparsity of \vec{β}. The optimization is performed over a space of 50,000 SNPs which are selected by rank ordering the p-values obtained from singlemarker regression of the phenotype against the SNPs. The details of this are described in the “Model Training Algorithm” section below.

Predictors are trained using a custom implementation of the LASSO algorithm which uses coordinate descent for a fixed value of A. In one embodiment, five (or another selected number) non-overlapping sets of cases and controls can be held back from the training set and used for the purposes of in-sample cross-validation. For each value of A, there is a particular predictor which is then applied to the cross-validation set, where the polygenic score is defined as (i labels the individual and j labels the SNP)

$${PGS}_{i} = {\sum }_{j} {X}_{ij} {β}_{j}^{*} . \qquad ( 2.2 )$$

A “polygenic score” may be thought of as comprising a simple measure built using results from single marker regression (e.g. GWAS), optionally combined with p-value thresholding, and a method to account for linkage disequilibrium. The inventors' use of penalized regression incorporates similar features—it favors sparse models (setting most effects to zero) in which the activated SNPs (those with non-zero effect sizes) are only weakly correlated to each other. A brief overview of the use of single marker regression for phenotypes studied here is set forth in the “Testing using Genetically Dissimilar Subgroups: Ancestry Out-Of-Sample Testing” section below.

To generate a specific value of the penalization A which defines the final predictor (for final evaluation on out-of-sample testing sets), a system employing the techniques described herein can find the λ that maximizes AUC in each cross-validation set, average them, then move one standard deviation in the direction of higher penalization (the penalization λ is progressively reduced in a LASSO regression). Moving one standard deviation in the direction of higher penalization errs on the side of parsimony. In this context, a more parsimonious model refers to one with fewer active SNPs. These values of λ* are reported in Table 1, but further analysis shows that tuning λ to a value that maximizes the testing set AUC tends to match λ* within error. This is explained in more detail in the “Model Training Algorithm” section below. The value of the phenotype variable y is simply 1 or 0 (for case or control status, respectively).

Scores can be turned into receiver operating characteristic (ROC) curves by binning and counting cases and controls at various reference score values. The ROC curves are then numerically integrated to get AUC curves. The precision of this procedure was tested by splitting ROC intervals into smaller and smaller bins. For several phenotypes this is compared to the rank-order (Mann-Whitney) exact AUC. The numerical integration, which was used to save computational time, gives AUC results accurate to ˜1%. This is the given accuracy at a specific number of cases and controls. As described in Sec. 3 the absolute value of AUC depends on the number of reported cases. For various AUC results the error is reported as the larger of either this precision uncertainty or the statistical error of repeated trials.

For the analysis of case-control phenotypes it is also possible to use logistic regression. Little to no difference in AUC or odds ratio results was found when comparing between linear and logistic regression methods used by the inventors to develop predictors. This might suggest that the data sets are highly constrained by the linear central region of the logistic function. Additionally, if a goal is to identify genomes corresponding to extreme outliers, a linear regression can be more conservative. Thus, in some instances, the inventors have found that a linear approach provides some unexpected advantages. In other instances, such as where higher order data exists, a logistic approach may have slightly higher likelihood of delivering better AUC and odds ratio results for a given predictor.

### Experiments and Results

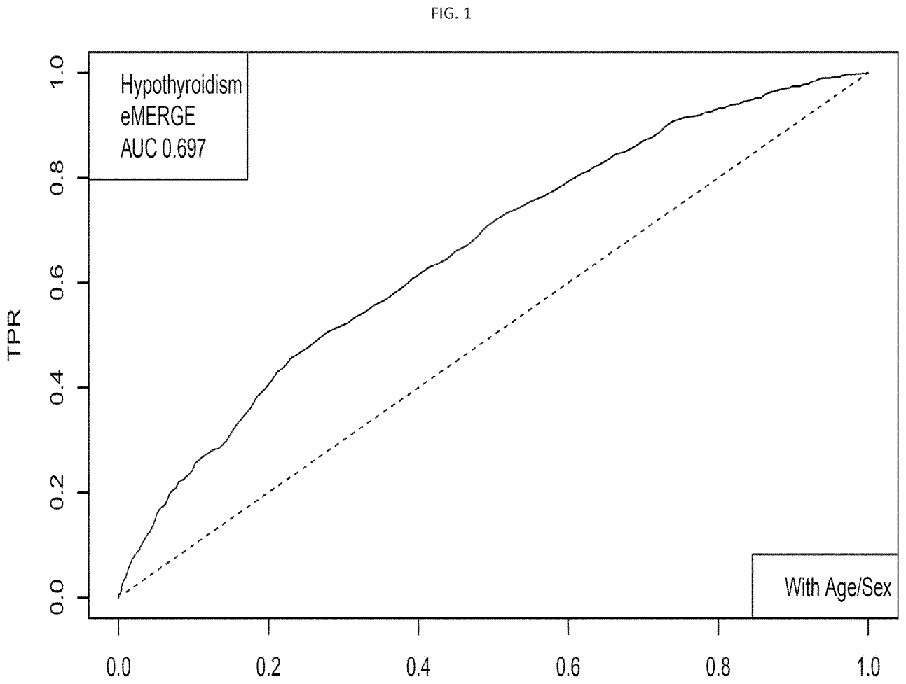

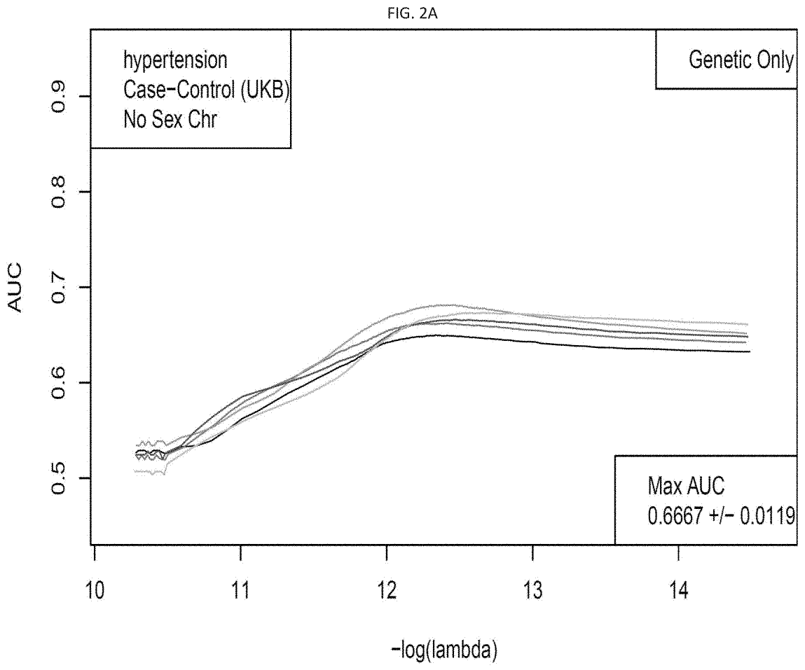

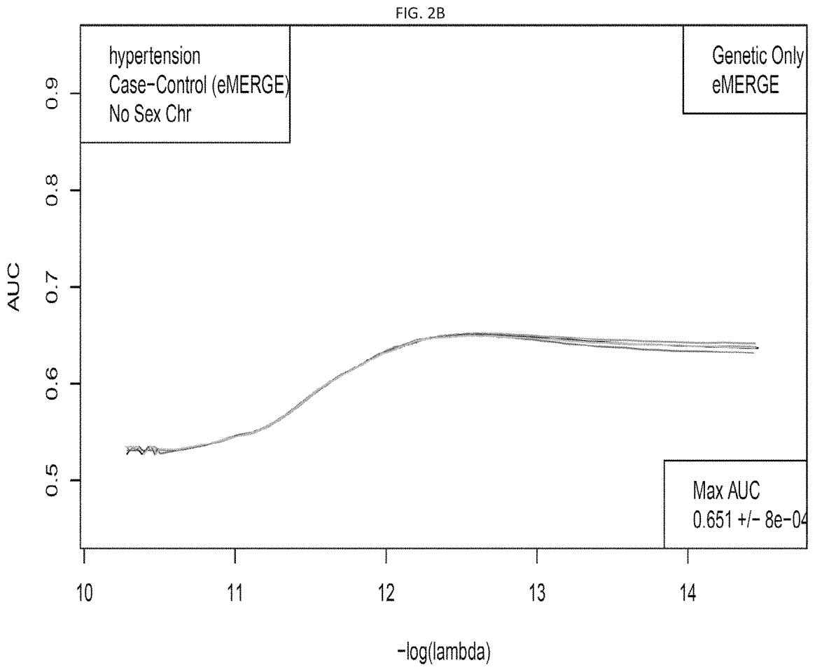

FIGS. 2A and 2B show the AUC score evaluation of a predictor built using a custom LASSO algorithm as described herein. The LASSO outputs can be used to build ROC curves, as shown in FIG. 1, and in turn produce AUCs and Odds Ratios. FIG. 2A uses the UK Biobank dataset and FIG. 2B uses the eMERGE dataset. Five non-overlapping sets of cases and controls are held back from the training set for the purposes of in-sample cross-validation. For each value of λ, there is a particular predictor which is then applied to the cross-validation set. The value of λ one standard deviation higher than the one which maximizes AUC on a cross-validation set is selected as the definition of the model. Models are additionally judged by comparing a non-parametric measure, Mann-Whitney data AUC, to a parametric prediction, Gaussian AUC.

Each training set builds a slightly different predictor. After each of the 5 predictors is applied to the in-sample cross-validation sets, each model is evaluated (by AUC) to select the value of λ which will be used on the testing set. For some phenotypes true out-of-sample data is available (i.e. eMERGE), while for other phenotypes ancestry out-of-sample (AOS) testing can be implemented using genetically dissimilar groups. This is described in Appendices C and D. An example of this type of calculation is shown in FIGS. 2A and 2B, where the AUC is plotted as a function of λ for Hypertension.

Table 1 below presents the results of similar analyses for a variety of disease conditions. The best AUC is listed for a given trait and the data set which was used to obtain that AUC. Training and validating is done using UKBB data from either direct calls or imputed data to match eMERGE. Testing is done with UKBB, eMERGE, or AOS as described in Secs. 2 and the “Testing using Genetically Dissimilar Subgroups: Ancestry Out-Of-Sample Testing” section below. Numbers in parenthesis are the larger of either a standard deviation from central value or numerical precision as described in Sec. 2. The variable λ* refers to the LASSO λ value used to compute AUC as described in Sec. 2.

TABLE 1

| Condition | Training Set | Test Set | AUC | Active SNPs | λ* |
|---|---|---|---|---|---|
| Hypothyroidism | Impute | UKBB | 0.705(0.009) | 3704(41) | 1.406e−06 |
| | | | | | (1.33e−7) |
| Hypothyroidism | Impute | eMERGE | 0.630(0.006) | 3704(41) | 1.406e−06 |
| | | | | | (1.33e−7) |
| Type 2 Diabetes | Impute | UKBB | 0.640(0.015) | 4168(61) | 6.93e−06 |
| | | | | | (1.73e−6) |
| Type 2 Diabetes | Impute | eMERGE | 0.633(0.006) | 4168(61) | 6.93e−06 |
| | | | | | (1.73e−6) |
| Hypertension | Impute | UKBB | 0.667(0.012) | 9674(55) | 4.46e−6 |
| | | | | | (4.86e−7) |
| Hypertension | Impute | eMERGE | 0.651(0.007) | 9674(55) | 4.46e−6 |
| | | | | | (4.86e−7) |
| Resistant | impute | eMERGE | 0.6861(0.001) | 9674(55) | 4.46e−6 |
| Hypertension | | | | | (4.86e−7) |
| Asthma | Calls | AOS | 0.632(0.006) | 3215(16) | 2.37e−6 |
| | | | | | (0.35e−6) |
| Type 1 Diabetes | Calls | AOS | 0.647(0.006) | 50(7) | 7.9e−7 |
| | | | | | (0.1e−7) |
| Breast Cancer | Calls | AOS | 0.582(0.006) | 480(62) | 3.38e−6 |
| | | | | | (0.05e−6) |
| Prostate Cancer | Calls | AOS | 0.6399(0.0077) | 448(347) | 3.07e−6 |
| | | | | | (0.08e−8) |
| Testicular | Calls | AOS | 0.65(0.02) | 19(7) | 1.42e−6 |
| Cancer | | | | | (0.04e−6) |
| Glaucoma | Calls | AOS | 0.606(0.006) | 610(114) | 8.69e−7 |
| | | | | | (0.71e−7) |
| Gout | Calls | AOS | 0.682(0.007) | 1010 (35) | 9.41e−7 |
| | | | | | (0.03e−7) |
| Atrial | Calls | AOS | 0.643(0.006) | 181(39) | 8.61e−7 |
| Fibrillation | | | | | (0.94e−7) |
| Gallstones | Calls | AOS | 0.625(0.006) | 981(163) | 1.01e−7 |
| | | | | | (0.02e−7) |
| Heart Attack | Calls | AOS | 0.591(0.006) | 1364(49) | 1.181e−6 |
| | | | | | (0.002e−7) |
| High | Calls | AOS | 0.628(0.006) | 3543(36) | 2.4e−6 |
| Cholesterol | | | | | (0.2e−6) |
| Malignant | Calls | AOS | 0.580(0.006) | 26(15) | 9.5e−7 |
| Melanoma | | | | | (0.8e−7) |
| Basal Cell | Calls | AOS | 0.631(0.006) | 76(22) | 9.9e−7 |
| Carcinoma | | | | | (0.3e−7) |

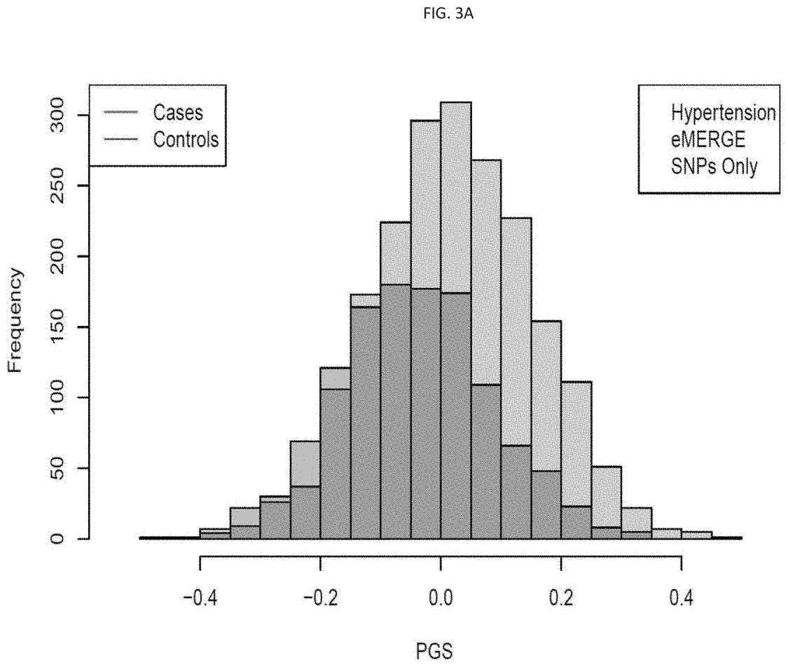

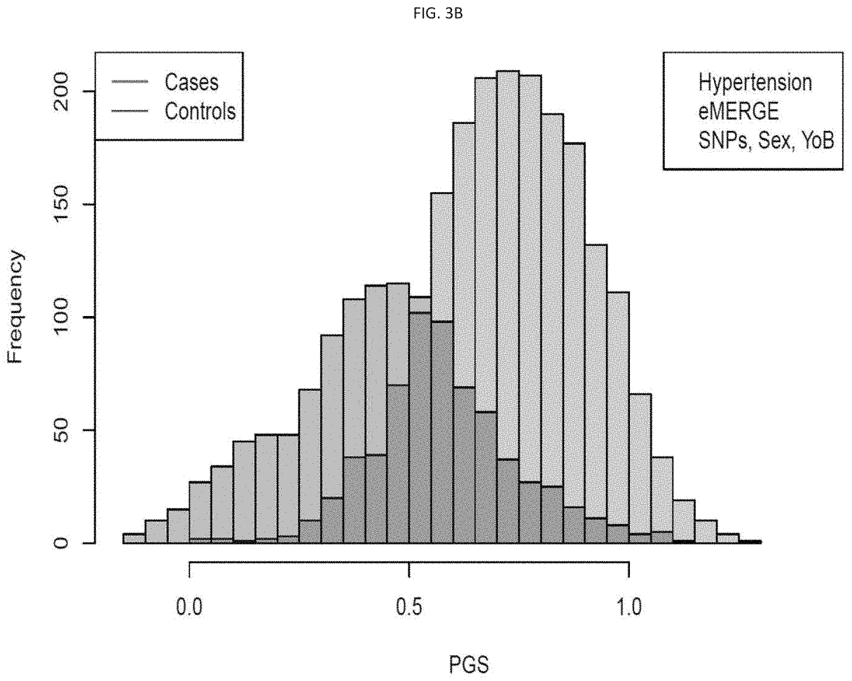

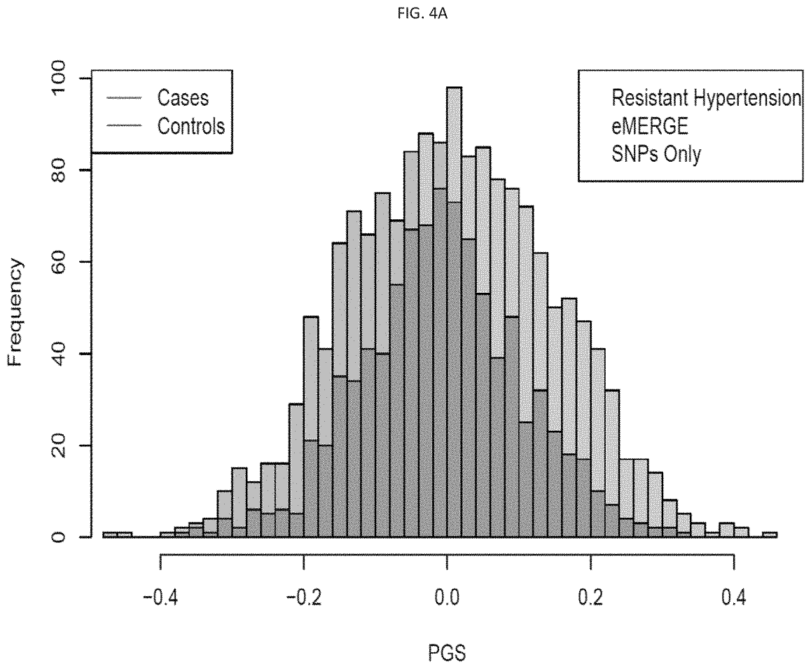

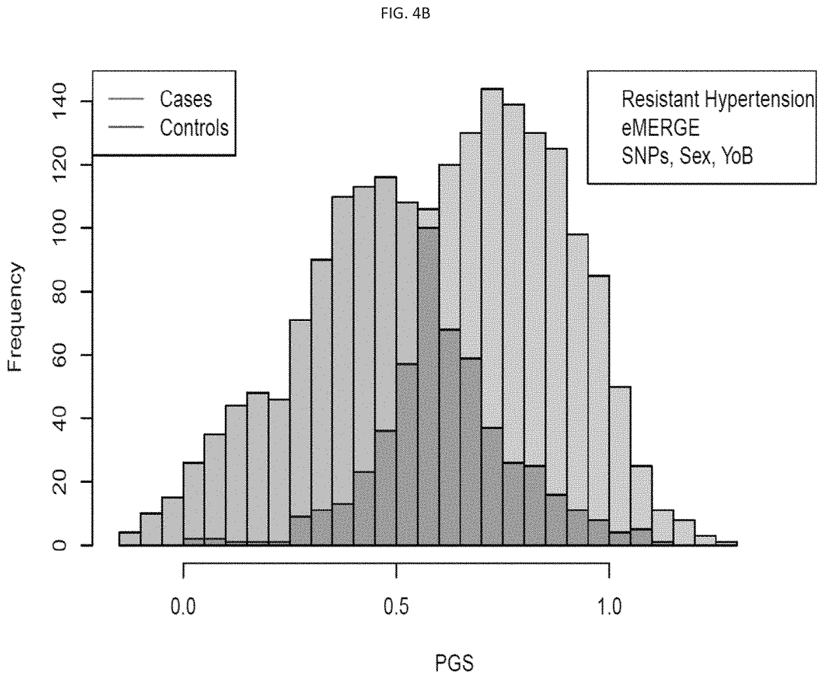

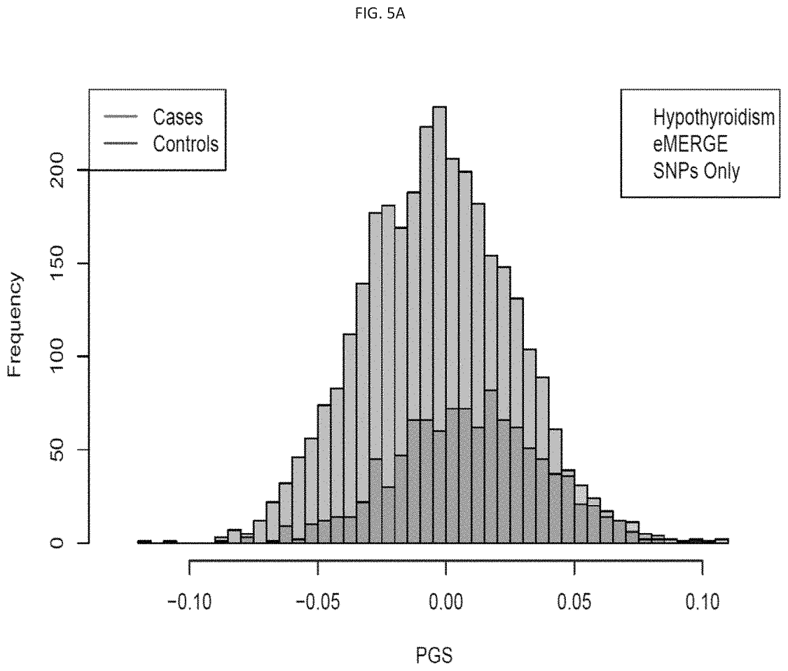

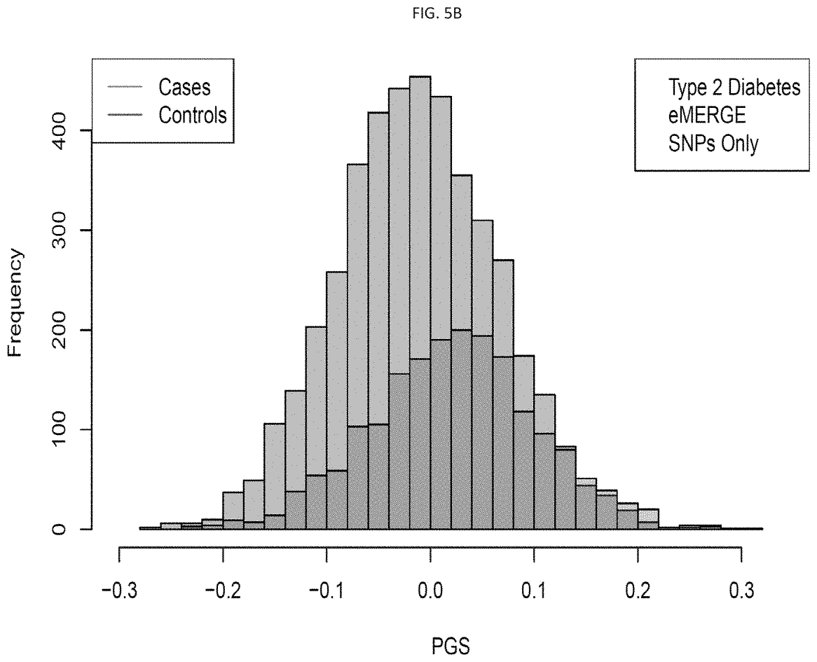

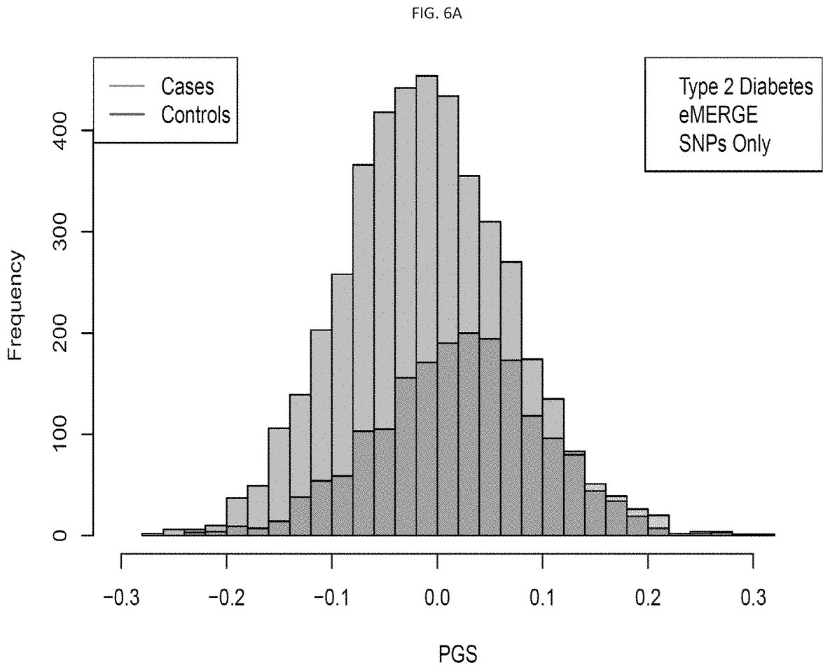

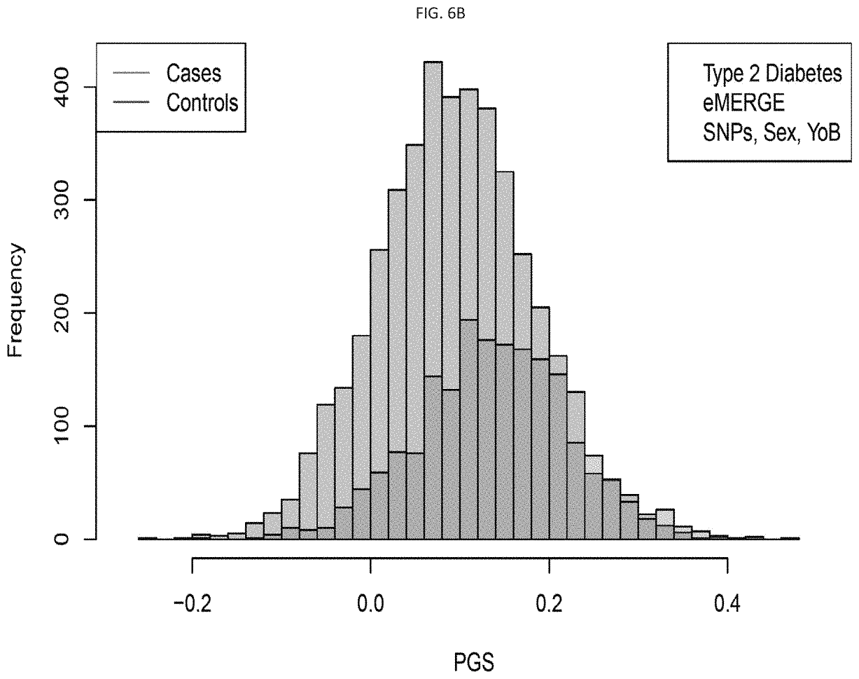

In FIGS. 3, 4, 5, and 6, the distributions of the polygenic score are shown for cases and controls drawn from the eMERGE dataset. In FIGS. 3A, 4A, 5A, and 6A, the distributions are obtained from performing LASSO on case-control data only, and FIGS. 3B, 4B, 5B, and 6B show an improved polygenic score (PGS) which includes effects obtained from separately regressing on sex and age. FIG. 3A is a graph of a distribution of PGS, cases and controls for Hypertension in the eMERGE dataset using SNPs alone. FIG. 3B is a graph of distribution of PGS, cases and controls for Hypertension in the eMERGE dataset using sex and age as regressors. FIG. 4A is a graph of distribution of PGS, cases and controls for Resistant Hypertension in the eMERGE dataset using SNPs alone. FIG. 4B is a graph of distribution of PGS, cases and controls for Resistant Hypertension in the eMERGE dataset using sex and age as regressors. FIG. 5A is a graph of distribution of PGS, cases and controls for Hypothyroidism in the eMERGE dataset using SNPs alone. FIG. 5B is a graph of distribution of PGS, cases and controls for Hypothyroidism in the eMERGE dataset using sex and age as regressors. FIG. 6A is a graph of distribution of PGS, cases and controls for type 2 diabetes in the eMERGE dataset using SNPs alone. FIG. 6B is a graph of distribution of PGS, cases and controls for type 2 diabetes in the eMERGE dataset using sex and age as regressors. The improved polygenic score is obtained as follows: regress the phenotype y=(1, 0) against sex and age, and then add the resulting model to the LASSO score. This procedure is reasonable since SNP state, sex, and age are independent degrees of freedom. In some cases, this procedure leads to vastly improved performance. The distribution of PGS among cases can be significantly displaced (e.g., shifted by a standard deviation or more) from that of controls when the AUC is high. At modest AUC, there is substantial overlap between the distributions, although the high-PGS population has a much higher concentration of cases than the rest of the population. Outlier individuals who are at high risk for the disease condition can therefore be identified by PGS score alone even at modest AUCs, for which the case and control normal distributions are displaced by, e.g., less than a standard deviation.

In Table 2 below, results from regressions on SNPs alone, sex and age alone, and all three combined are compared. Performance for some traits is significantly enhanced by inclusion of sex and age information.

For example, Hypertension is predicted very well by age+sex alone compared to SNPs alone whereas Type 2 Diabetes is predicted very well by SNPs alone compared to age+sex alone. In all cases, the combined model outperforms either individual model.

TABLE 2

| | | Age + | Genetic | Age + Sex + |
|---|---|---|---|---|
| Condition | Testset | Sex | Only | genetic |
| Hypertension | UKBB | 0.638 (0.018) | 0.667 (0.012) | 0.717 (0.007) |
| Hypothyroidism | UKBB | 0.695 (0.007) | 0.705 (0.009) | 0.783 (0.008) |
| Type 2 Diabetes | UKBB | 0.672 (0.009) | 0.640 (0.015) | 0.651 (0.013) |
| Hypertension | eMERGE | 0.818 (0.008) | 0.651 (0.007) | 0.851 (0.009) |
| Resistant | eMERGE | 0.817 (0.008) | 0.686 (0.007) | 0.864 (0.009) |
| Hypertension | | | | |
| Hypothyroidism | eMERGE | 0.643 (0.006) | 0.630 (0.006) | 0.697 (0.007) |
| Type 2 Diabetes | eMERGE | 0.565 (0.006) | 0.633 (0.006) | 0.651 (0.007) |

The results presented above entailed on predictions built on the autosomes alone (i.e. SNPs from the sex chromosomes are not included in the regression). However, given that some conditions are predominant in one sex over the other, for some traits or diseases there may be a nontrivial effect coming from the sex chromosomes. For instance, 85% of Hypothyroidism cases in the UK Biobank are women. Accordingly, one aspect of the systems described herein is to implement systems that take gender correlation of diseases into account, and automatically generate and compare predictor models with and without taking into account SNPs from the sex chromosomes.

In Table 3 the results (AUCs) from including the sex chromosomes in a predictor generation technique are compared to using only the autosomes. The differences found in terms of AUC are negligible for the diseases identified below, suggesting that variation among common SNPs on the sex chromosomes does not have a large effect on Hypothyroidism, Type 2 Diabetes, Hypertension, and Resistant Hypertension risk. A similarly negligible change was found when including sex chromosomes for AOS testing. All conditions were tested on eMERGE using SNPs as the only covariate. Thus, for these diseases, a system implementing the techniques disclosed herein might determine the inclusion of the sex chromosome in future updates of the predictor models would be unnecessary until some threshold number of additional records is added to appropriate subsets or collations of the training genomic dataset (whereupon the comparison could be re-performed).

TABLE 3

| Condition | With Sex Chr | No Sex Chr |
|---|---|---|
| Hypothyroidism | 0.6302 (0.0012) | 0.6300 (0.0012) |
| Type 2 Diabetes | 0.6377 (0.0018) | 0.6327 (0.0018) |
| Hypertension | 0.6499 (0.0008) | 0.6510 (0.0008) |
| Resistant Hypertension | 0.6845 (0.001) | 0.6861 (0.001) |

FIGS. 3, 4, 5, and 6 suggest that case and control populations can be approximated by two overlapping normal distributions. Under this assumption, one can relate AUC directly to the means and standard deviations of the case and control populations. If two normal distributions with means μ₁, μ₀ and standard deviations σ₁, σ₀ are assumed for cases and controls (i=1, 0 respectively below), the AUC can be explicitly calculated via Equation 3.1:

$$f ( x , {μ}_{i} , {σ}_{i} ) = \frac{1}{\sqrt{2 π {σ}_{i}^{2}}} exp ( - \frac{1}{2} {( \frac{x - {μ}_{i}}{{σ}_{i}} )}^{2} ) \\ Φ ( t ) = {\int }_{- ∞}^{t} dxf ( x , 0 , 1 ) \\ AUC = Φ ( \frac{{μ}_{1} - {μ}_{0}}{\sqrt{{σ}_{1}^{2} + {σ}_{0}^{2}}} ) \qquad ( 3.1 )$$

The details of this approach are in the “Analytic AUC and Risk” section below. Under the assumption of overlapping normal distributions, the following odds ratio OR(z) can be computed as a function of PGS. OR(z) is defined as the ratio of cases to controls for individuals with PGS≥z to the overall ratio of cases to controls in the entire population. In Equation 3.2 below, 1=cases, 0=controls.

$$OR ( z ) = \frac{{\int }_{z}^{∞} dx ( {n}_{1} {f}_{1} ( x ) ) / {\int }_{z}^{∞} dx ( {n}_{0} {f}_{0} ( x ) )}{{n}_{1} / {n}_{0}} = \frac{1 - Φ ( \frac{z - {μ}_{1}}{{σ}_{1 }} )}{1 - Φ ( \frac{z - {μ}_{0}}{{σ}_{0}} )} \qquad ( 3.2 )$$

The means and standard deviations for cases and controls are computed using the PGS distribution defined by the best predictor (by AUC) in the eMERGE dataset. The AUC and OR predicted under the assumption of displaced normal distributions can then be compared with the actual AUC and OR calculated directly from eMERGE data.

AUC results are shown in Table 4, where the statistics for predictors trained on SNPs alone are shown. Mean μ and standard deviation a for PGS distributions are given for cases and controls, using predictors built from SNPs only and trained on case-control status alone. Predicted AUC from assumption of displaced normal distributions and actual AUC are also given. Table 5 shows the same statistics as Table 4 but for predictors trained on SNPs, sex, and age. Mean μ and standard deviation a for PGS distributions of cases and controls are given, using predictors built from SNPs, sex, and age, and trained on case-control status alone. Predicted AUC from assumption of displaced normal distributions and actual AUC are also given.

TABLE 4

| | Hypothyroidism | Type 2 Diabetes | Hypertension | Res HT |
|---|---|---|---|---|
| μ_{case} | 0.0093 | 0.0271 | 0.0240 | 0.0392 |
| μ_{control} | −0.0038 | −0.0141 | −0.0470 | −0.0448 |
| σ_{case} | 0.0284 | 0.0901 | 0.1343 | 0.1270 |
| σ_{control} | 0.0276 | 0.0866 | 0.1281 | 0.1219 |
| N_{cases}/N_{controls} | 1,084/3,171 | 1,921/4,369 | 2,035/1,202 | 1,358/1,202 |
| AUC_{pred} | 0.630 (0.006) | 0.629 (0.006) | 0.649 (0.006) | 0.683 (0.007) |
| AUC_{actual} | 0.630 (0.006) | 0.633 (0.006) | 0.651 (0.007) | 0.686 (0.006) |

TABLE 5

| | Hypothyroidism | Type 2 Diabetes | Hypertension | Res HT |
|---|---|---|---|---|
| μ_{case} | 0.1516 | 0.1431 | 0.7377 | 0.7525 |
| μ_{control} | 0.1185 | 0.0924 | 0.4375 | 0.4366 |
| σ_{case} | 0.0437 | 0.0948 | 0.1829 | 0.1830 |
| σ_{control} | 0.0474 | 0.0943 | 0.2250 | 0.2258 |
| N_{cases}/N_{controls} | 1,035/3,047 | 1,921/4,369 | 2,000/1,196 | 1,331/1,196 |
| AUC_{pred} | 0.696 (0.007) | 0.648 (0.006) | 0.850 (0.009) | 0.862 (0.009) |
| AUC_{actual} | 0.697 (0.007) | 0.651 (0.007) | 0.852 (0.009) | 0.864 (0.009) |

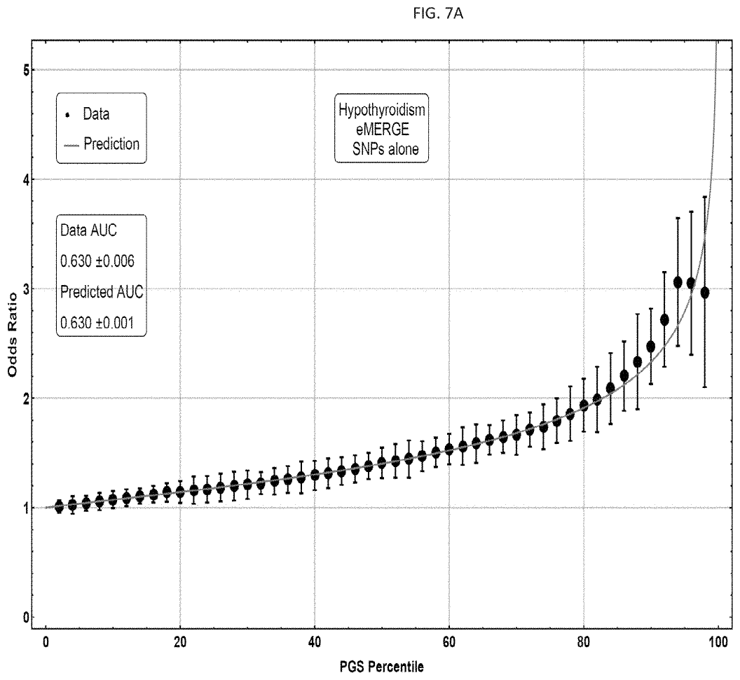

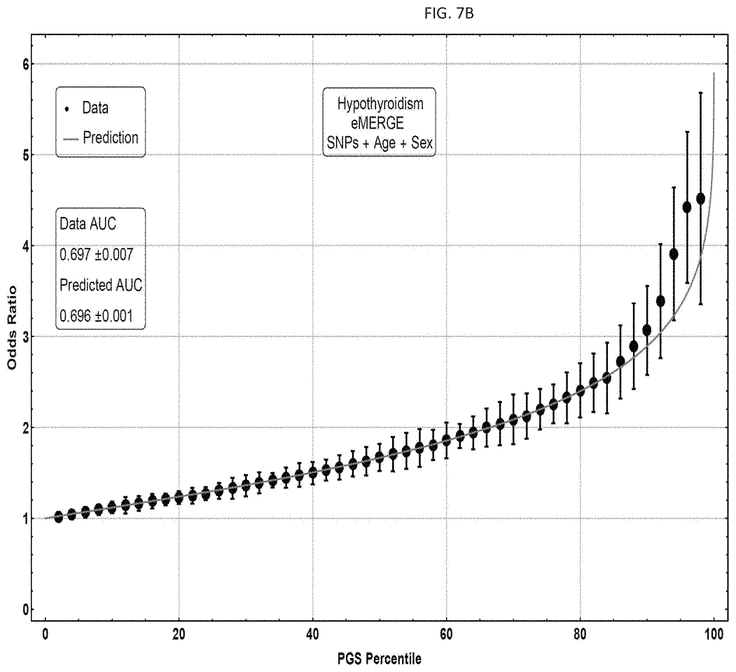

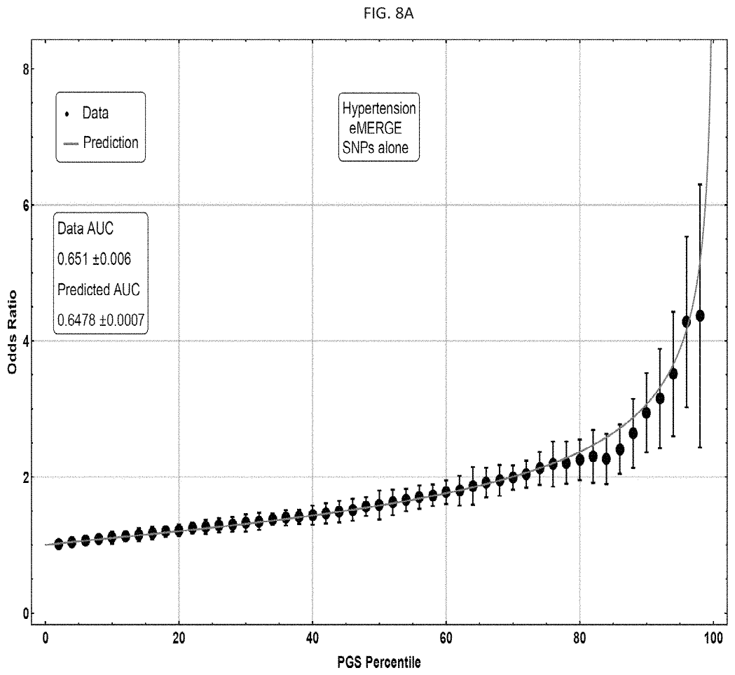

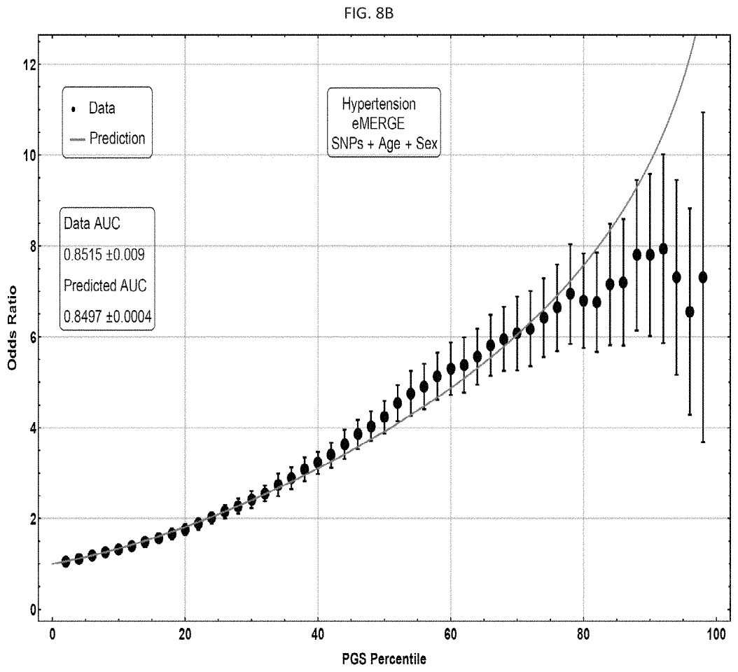

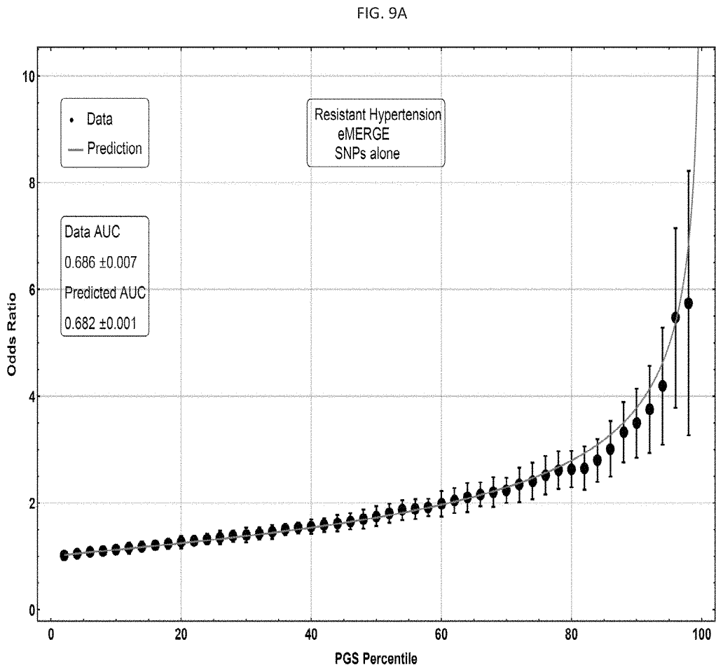

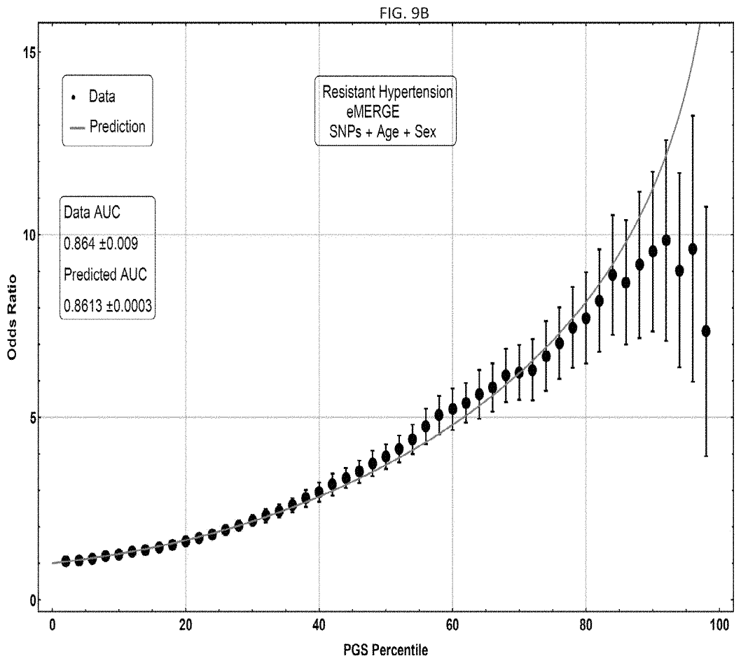

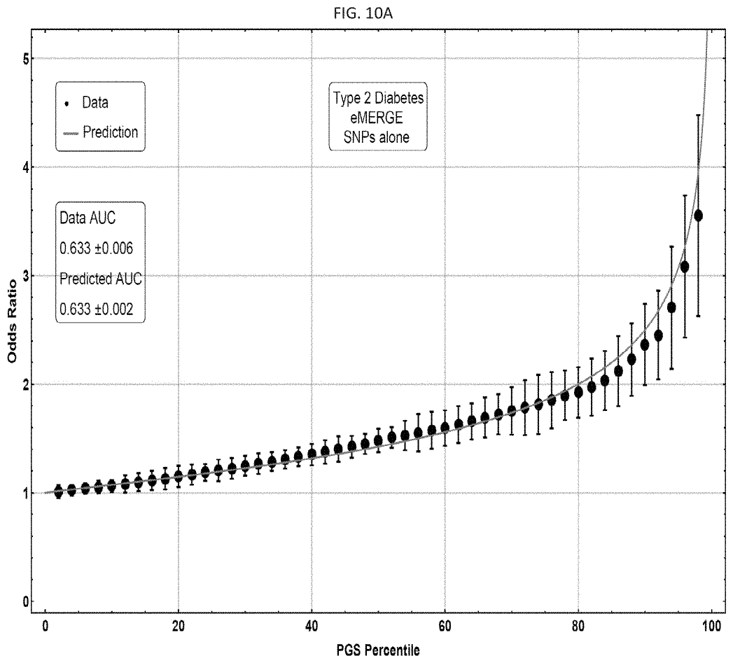

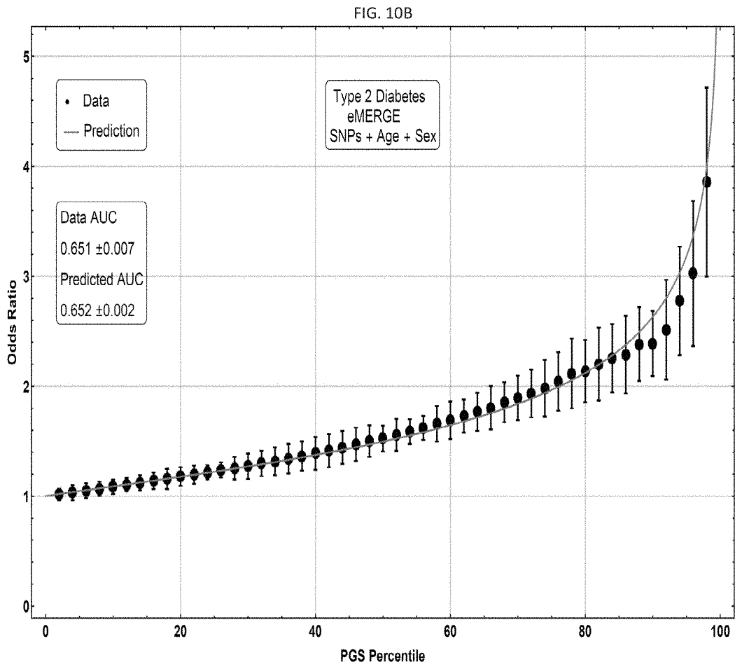

The results for odds ratios as a function of PGS percentile for several conditions are shown in FIGS. 7, 8, 9, and 10. Each figure shows the results when 1) performing the modified LASSO technique disclosed herein on case-control data only and 2) performing the LASSO technique on the same data but adding sex and age. The red line is what one obtains using the assumption of displaced normal distributions (i.e., Equation 3.2). FIG. 7A is a graph of odds ratio between upper percentile in PGS and total population prevalence in eMERGE for Hypothyroidism using SNPs alone. FIG. 7B is a graph of odds ratio between upper percentile in PGS and total population prevalence in eMERGE for Hypothyroidism using SNPs with sex and age as covariates. FIG. 8A is a graph of odds ratio between upper percentile in PGS and total population prevalence in eMERGE for Hypertension using SNPs alone. FIG. 8B is a graph of odds ratio between upper percentile in PGS and total population prevalence in eMERGE for Hypertension using SNPs with sex and age as covariates. FIG. 9A is a graph of odds ratio between upper percentile in PGS and total population prevalence in eMERGE for Resistant Hypertension using SNPs alone. FIG. 9B is a graph of odds ratio between upper percentile in PGS and total population prevalence in eMERGE for Resistant Hypertension using SNPs with sex and age as covariates. FIG. 10A is a graph of odds ratio between upper percentile in PGS and total population prevalence in eMERGE for Type 2 Diabetes using SNPs alone. FIG. 10B is a graph of odds ratio between upper percentile in PGS and total population prevalence in eMERGE for Type 2 Diabetes using SNPs with sex and age as covariates. Overall there is good agreement between directly calculated odds ratios and the red line.

Odds ratio error bars come from 1) repeated calculations using different training sets and 2) by assuming that counts of cases and controls are Poisson distributed. (This increases the error bar or estimated uncertainty significantly when the number of cases in a specific PGS bin is small.)

In the analysis performed by the inventors, it was tested whether altering the regressand (phenotype y) to some kind of residual based on age and sex could improve the genetic predictor. To start, in all cases, y=1, 0 for case or control respectively and then three different regressands were used:

$$\begin{matrix}{y}^{′} = y ( y = 1 , 0 ) ; CC status alone & ( 3.3 ) \\ {y}^{′} = y - ( {β}_{0} + {β}_{S} S + {β}_{Age} Age ) ; Modification 1 & ( 3.4 ) \\ {y}^{′} = \frac{y - {μ}_{M / F}}{{σ}_{M / F}} - ( {β}_{0} + {β}_{Age} Age ) ; Modification 2 & ( 3.5 )\end{matrix}$$

For each case, both including and excluding the sex chromosomes during the regression was tested. As with the previous results, the best prediction accuracy is not appreciably altered if training is done on the autosomes alone. The results are given in Table 6. Prediction results are given for three types of regressands. All results are on eMERGE and show results for using SNPs, Age, Sex and combinations of such.

TABLE 6

| | CC | Mod | Mod |
|---|---|---|---|
| Condition | Status | 1 | 2 |

Hypothyroidism

| SNPs | 0.6300 | (0.0012) | 0.6046 (0.0025) | 0.6177 | (0.0042) |
|---|---|---|---|---|---|
| alone | | | | | |
| Age/Sex | 0.6430 | | | | |
| Alone | | | | | |
| With | 0.6966 | (0.0009) | 0.6489 (0.0173) | 0.6884 | (0.0021) |
| Age/Sex | | | | | |

Type 2 Diabetes

| SNPs | 0.6327 | (0.0018) | 0.6378 (0.0018) | 0.6327 | (0.0018) |
|---|---|---|---|---|---|
| alone | | | | | |
| Age/Sex | 0.5654 | | | | |
| Alone | | | | | |
| With | 0.651 | (0.0014) | 0.6283 (0.0039) | 0.651 | (0.0014) |
| Age/Sex | | | | | |

Hypertension

| SNPs | 0.651 | (0.0008) | 0.6495 (0.0004) | 0.6497 | (0.0005) |
|---|---|---|---|---|---|
| alone | | | | | |
| Age/Sex | 0.8180 | | | | |
| Alone | | | | | |
| With | 0.8518 | (0.0003) | 0.8519 (0.0003) | 0.8516 | (0.0001) |
| Age/Sex | | | | | |

The distributions in FIGS. 3-5 appear Gaussian, and were further tested against a normal distribution. Atrial Fibrillation and Testicular cancer represent respectively the best and worst fits to Gaussians. For control groups, results were similar for all phenotypes. For example assuming “Sturge's Rule” for the number of bins, Atrial Fibrillation controls lead to χ_{dof}²=5,359.29=56,772 with a p-value 7×10\hat{ }-1013 when tested against a Gaussian distribution. For cases, the inventors also found extremely good fits. Again, Atrial Fibrillation cases lead to χ_{dof}²=35.181=418 and p-value 0.0192. Even for phenotypes with very few cases the inventors found very good fits. For Testicular Cancer cases the inventors found χ_{dof}²=35.1429/89 and p-value 1.18×10⁻⁴. For predicted AUCs and Odds Ratios using Eqs. (3.1) & (3.2) the inventors found very little difference between using means and standard deviations from empirical data sets or using fits to Gaussians.

As more data becomes available for training a given predictor (e.g., data records are added to a dataset incrementally as users join a reporting service, or healthcare records of various health systems are linked), it is evident from the inventors' work that prediction strength (e.g., AUC) will increase. Accordingly, another aspect of the systems that can be built according to the techniques disclosed herein is that the systems may automatically acquire and pre-process more data records as they become available. After each record is added to a dataset (or periodically after a set number or threshold of records is added) the systems can periodically retrain their predictor models and can even reach tipping points of records at which predictors for certain rare traits or disease can become available.

By way of further explanation behind the confidence that utilizing additional training data will continue to yield better accuracy, the inventors conducted several tests that revealed this to be the case. Based on estimated heritability, the inventors determined that several of the predictors set forth above from the foregoing example study are relatively far from maximum possible AUCs (e.g., the point at which adding further training data would yield diminishing or insignficiant results), such as: type 2 diabetes (0.94), coronary artery disease (0.95), breast cancer (0.89), prostate cancer (0.90), and asthma (0.88). Improvements in accuracy as a function of additional training data were investigated with sample size by varying the number of cases used in training. For Type 2 Diabetes and Hypothyroidism, predictors were trained with 5 random sets of 1k, 2k, 3k, 4k, 6k, 8k, 10k, 12k, 14k, and 16k cases (all with the same number of controls). For Hypertension, predictors were trained using 5 random sets of 1k, 10k, 20k, . . . , up to 90k cases. For each predictor, the previously generated best predictors which used all cases except the 1000 held back for cross-validation were included. These predictors are then applied to the eMERGE dataset and the maximum AUC is calculated.

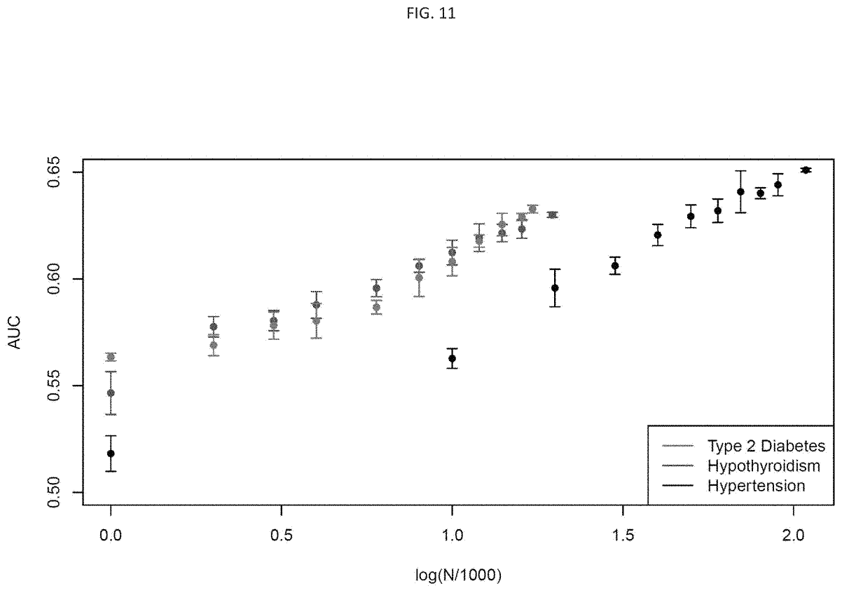

In FIG. 11 the average maximum AUC among the 5 training sets is plotted against the log of the number of cases (in thousands) used in training. FIG. 11 is a graph of a graph of maximum AUC on out-of-sample testing set (eMERGE) as a function of the number of cases (in thousands) included in training for type 2 diabetes, Hypothyroidism and Hypertension. Note that in each situation, as the number of cases increases, so does the average AUC. For each disease condition, the AUC increases roughly linearly with log N as the maximum number of cases available is approached. The rate of improvement for Type 2 Diabetes appears to greater than for Hypertension or Hypothyroidism, but in all cases there is no sign of diminishing returns.

By extrapolating this linear trend, the value of AUC obtainable can be projected using a future cohort with a larger number of cases. In this work, Type 2 Diabetes, Hypothyroidism, and Hypertension predictors were trained using 17k, 20k and 108k cases, respectively.

If, for example, cohorts were assembled with 100k, 100k and 500k cases, then the linear extrapolation suggests AUC values of 0.70, 0.67 and 0.71 respectively. This corresponds to 95 percentile odds ratios of approximately 4.65, 3.5, and 5.2. In other words, it is reasonable to project that future predictors will be able to identify the 5 percent of the population with at least 3-5 times higher likelihood for these conditions than the general population. As discussed below, the ability to identify these individuals has important clinical implications. Thus, various systems and applications are disclosed herein that leverage these predictors for purposes ranging from simply information patients of their risk levels, to giving guidance to healthcare professionals, to various health insurance actuarial and marketing improvements.

The three traits above were focused on because out of sample can be tested using eMERGE. However, using the Ancestry Out of Sample (AOS) method, similar projections can be made for diseases which may 1) be more clinically actionable or 2) show more promise for developing well separated cases and controls. AOS testing was performed while varying the number of cases included in training for Type 1 Diabetes, Gout, and Prostate Cancer. Predictors were trained using all but 500, 1000, and 1500 cases and fit the maximum AUC to log(N/1000) to estimate AUC in hypothetical new datasets. For Type 1 Diabetes, training was performed with 2234, 1734 and 1234 cases —which achieve AUC of 0.646, 0.643, 0.642. For Gout training was performed with 5503, 5003 and 4503 cases achieving AUC of 0.0.681, 0.676, 0.0.673. For Prostate Cancer, training was performed with 2758, 2258, 1758 cases achieving AUC of 0.0.633, 0.628, 0.609. A linear extrapolation to 50k cases of Prostate Cancer, Gout, and Type 1 Diabetes suggests that new predictors could achieve AUCs of 0.79, 0.76 and 0.66 (respectively) based solely on genetics. Such AUCs correspond to odds ratios of and 11, 8, and 3.3 (respectively) for 95th percentile PGS score and above.

### Methodologies and System Design

Genotype Quality Control

The main dataset used for training the examples set forth above was the 2018 release of the UK Biobank (the 2018 version corrected some issues with imputation, included sex chromosomes, etc). In the example experiments above, analysis was performed on records of genetically British individuals (as defined using ancestry principal component analysis performed by UK Biobank). In 2018, the UK Biobank (UKBB) re-released the dataset representing approximately 500,000 individuals genotyped on two Affymetrix platforms—approximately 50,000 samples on the UKB BiLEVE Axiom array and the remainder on the UKB Biobank Axiom array. The genotype information was collected for 488,377 individuals for 805,426 SNPs which were then subsequently imputed to a much larger number of SNPs.

The imputed data set was generated using the set of 805,426 raw markers using the Haplotype Reference Consortium and UK10K haplotype resources. After imputation and initial QC, there were a total of 97,059,328 SNPs and 487,409 individuals. From this imputed data, further quality control was performed using Plink version 1.9. For out-of-sample testing of polygenic risk scores, imputed UK Biobank SNPs which survived the prior quality control measures, and are also present in a second dataset from the Electronic Medical Records and Genomics (eMERGE) study are kept. After keeping SNPs which are common to both the UK Biobank and eMERGE, 557,595 SNPs remained. Additionally SNPs and samples which had missing call rates exceeding 3% were excluded and SNPs with minor allele frequency below 0.1% were also removed so to avoid rare variants. This resulted in 468,514 SNPs and, upon restricting to genetically British, 408,954 people.

It can be understood that a similar method can be applied to other datasets. For example, in one embodiment, a dataset from UKBB or eMERGE might be combined with a dataset acquired by other means (e.g., from a health care system, from an online ancestry company, or acquired one-by-one from individual customers). Or an entirely non-public dataset might be used, without any data from UKBB, eMERGE, or similar sets. Control records can be withheld and processed as set forth above, from any dataset or merged datasets. Once an initial training dataset has been assembled and a set of SNPs determined, that set of SNPs can be used for purposes of pre-processing, culling, and quality checking future records that are acquired (to assess whether they could potentially be added to the dataset for future updating and refinement of the predictor models. As discussed above, some records may be suitable for use in refining specific predictors for particular traits or conditions (e.g., Type II Diabetes) but not others (e.g., Hypertension). A pre-processing module in accordance with the disclosure herein will make those determinations based upon the SNPs and phenotype data employed by each generated predictor.

Phenotype Quality Control

In addition to a QC process to determine whether sufficient genomic data exists in a given record or set of records, a comparable process is used to assess the strength of phenotypic data. A pre-processing module would also, therefore, be reviewing the types of diagnosis, outcome, etc. metadata that a genomic dataset contains.

Case/Control information for each given disease condition or trait is assessed. In many cases, this is a relatively simplistic check: to create a predictor model for height, data from genomic datasets that contain height measurements should be used. In other instances, a more nuanced approach should be taken.

In the experiments noted above, three case-control conditions were considered as examples, given they were disease conditions recorded and present in both the UK Biobank and eMERGE datasets: Hypothyroidism, Type 2 Diabetes, and Hypertension. In the instance of generating a predictor model for Type 2 Diabetes, for example, records were identified as comprising the “Cases.” To select Type 2 Diabetes cases in UKBB, individuals can be identified based on a doctor's diagnosis using the fields Diagnoses primary ICD10 or Diagnoses secondary ICD10. Specifically, any individual with ICD10 code E11.0-E11.9 (Non-insulin-dependent diabetes mellitus) in the Main Diagnosis or Secondary Diagnosis field. For training only, younger individuals who may still yet develop Type 2 Diabetes were to be excluded, so controls were selected using individuals in the remainder of the UKBB population not identified as cases and born on 1945 or earlier. This resulted in 18,194 cases and 108,726 controls among genetically British individuals. This example serves to demonstrate that for some disease conditions, the pre-processing module can be programmed to cull records by more than simply the presence of a database field containing a diagnosis.

For both Hypertension and Hypothyroidism, the field “Non-Cancer Illness Code (self-reported)” was used to identify cases and controls. As in the case of Type 2 diabetes, younger individuals were excluded as controls for Hypertension. This was not required for Hypothyroidism. Specifically, cases were identified by anyone with the code “1065” (Hypertension) in “Noncancer illness code (self-reported)” and the remainder of the UKBB population who were born before 1950 were selected as controls. This resulted in 109,662 cases and 140,689 controls for Hypertension. For Hypothyroidism, cases were identified by anyone with the code “1226” (Hypothyroidism/Myxoedema) in “Non cancer illness code (self-reported)” and the remainder of the UKBB population was used as a control. This resulted in 20,656 cases and 388,298 controls for Hypothyroidism.

For some phenotypes, it may be the case that true out of sample data is not available. In those instances, the Ancestry Out-of-Sample (AOS) based testing procedures can be used, for example in line with the “Testing using Genetically Dissimilar Subgroups: Ancestry Out-Of-Sample Testing” section below. In the inventors' experiments, data for the following disease conditions was pre-processed in this fashion: Gout, Testicular Cancer, Gallstones, Breast Cancer, Atrial Fibrillation, Glaucoma, Type 1 Diabetes, High Cholesterol, Asthma, Basal Cell Carcinoma, Malignant Melanoma, Prostate Cancer, and Heart Attack. All conditions were identified using the fields “Non cancer illness code (self-reported)”, “Cancer code (self-reported)” and “Diagnoses primary ICD10” or “Diagnoses secondary ICD10”.

Cases and controls of the following non-cancer illnesses were identified using the field “Non-Cancer Illness Code (self-reported)”: Gout, Gallstones, Atrial Fibrillation, Glaucoma, High Cholesterol, Asthma and Heart Attack. Cases for a specific non-cancer illness were identified as any individual with the following codes, and the remaining population are selected as a controls: Gout 1466, Gallstones 1162, Atrial Fibrillation 1471, Glaucoma 1277, High Cholesterol 1473, Asthma 1111, Heart Attack 1075. Cases and controls of the following cancer conditions were extracted from the field “Cancer Code (self-reported)”: Testicular Cancer, Prostate Cancer, Breast Cancer, Basal Cell Carcinoma and Malignant Melanoma. Specifically, cases were identified as any individual with the following codes, and controls are the remainder of the population: Testicular Cancer 1045, Breast Cancer 1002, Basal Cell Carcinoma 1061, Malignant Melanoma 1059, Prostate Cancer 1044. To select Type 1 Diabetes cases in UKBB, individuals were identified based on a doctor's diagnosis using the fields “Diagnoses primary ICD10” or “Diagnoses secondary ICD10”. Specifically, any individual with ICD10 code E10.0-E10.9 (Insulin-dependent diabetes mellitus) in the Main Diagnosis or Secondary Diagnosis field.

After identifying cases and controls in the whole UKBB population, the training set was restricted to “Genetically British” and the testing set to self-reported non-genetically-British Caucasian individuals. The number of cases and controls identified in this manner are listed in Table 7. Table 7 includes a number of cases and controls in training and testing sets for psuedo out-of-sample testing. Conditions with (*) are trained and tested only on a single sex.

TABLE 7

| | Cases | Controls | Cases | Controls |
|---|---|---|---|---|
| Condition | (train) | (train) | (test) | (test) |
| Gout | 6,003 | 395,842 | 811 | 56,383 |
| Gallstones | 7,022 | 394,823 | 936 | 56,258 |
| Atrial Fibrillation | 3,502 | 398,343 | 420 | 56,774 |
| Glaucoma | 4,609 | 397,236 | 577 | 56,617 |
| Type 1 Diabetes | 2,734 | 399,111 | 388 | 56,806 |
| High Cholesterol | 52,398 | 349,447 | 6,937 | 50,257 |
| Asthma | 47,237 | 354,608 | 6,655 | 50,539 |
| Basal Cell Carcinoma | 4,132 | 397,713 | 577 | 56,617 |
| Malignant Melanoma | 3,301 | 398,544 | 444 | 56,750 |
| Heart Attack | 9,657 | 398,544 | 1,347 | 55,847 |
| Prostate Cancer * | 3,258 | 181,518 | 379 | 24,733 |
| Breast Cancer * | 9,177 | 207,892 | 1,344 | 30,738 |
| Testicular Cancer * | 716 | 184,060 | 91 | 25,021 |

Table 8 is included below which outlines what fraction of cases and controls are male or female. The mean year of birth is also included for male/female cases/controls. The fraction of cases and controls and mean year of birth by sex for pseudo out-of-sample testing are shown. Traits with (*) are trained and tested only on a single sex.

TABLE 8

| | | | Mean Birth | Mean Birth |
|---|---|---|---|---|
| | % | % | Year | Year |
| | Female | Female | (Female) | (Female) |
| Condition | Cases | Controls | Cases | Controls |
| Gout | 7.35 | 54.98 | 1946.4 | 1951.5 |
| Gallstones | 77.59 | 53.87 | 1949.0 | 1951.6 |
| Atrial Fibrillation | 31.06 | 54.48 | 1945.8 | 1951.5 |
| Glaucoma | 46.91 | 54.36 | 1946.5 | 1951.5 |
| Type 1 Diabetes | 41.45 | 54.36 | 1950.4 | 1951.5 |
| High Cholesterol | 42.98 | 55.95 | 1946.7 | 1952.0 |
| Asthma | 57.48 | 53.85 | 1952.0 | 1951.4 |
| Basal Cell Carcinoma | 58.40 | 54.23 | 1948.5 | 1951.5 |
| Malignant Melanoma | 58.88 | 54.24 | 1949.6 | 1951.5 |
| Heart Attack | 19.68 | 55.11 | 1945.9 | 1951.5 |
| Prostate Cancer * | 0.0 | 0.0 | NA | NA |
| Breast Cancer * | 100.0 | 100.0 | 1946.0 | 1951.6 |
| Testicular Cancer * | 0.0 | 0.0 | NA | NA |
| | | | Mean Birth | Mean Birth |
| | % | % | Year | Year |
| | Male | Male | (Male) | (Male) |
| Condition | Cases | Controls | Cases | Controls |
| Gout | 92.65 | 45.02 | 1948.5 | 1951.2 |
| Gallstones | 22.41 | 46.13 | 1947.5 | 1951.1 |
| Atrial Fibrillation | 68.94 | 45.52 | 1946.4 | 1951.1 |
| Glaucoma | 53.09 | 45.64 | 1946.5 | 1951.1 |
| Type 1 Diabetes | 58.55 | 45.64 | 1949.0 | 1951.1 |
| High Cholesterol | 57.02 | 44.05 | 1947.3 | 1951.8 |
| Asthma | 42.52 | 46.15 | 1952.0 | 1951.0 |
| Basal Cell Carcinoma | 41.60 | 45.77 | 1947.4 | 1948.5 |
| Malignant Melanoma | 41.12 | 45.76 | 1948.2 | 1951.1 |
| Heart Attack | 80.32 | 44.89 | 1946.2 | 1945.9 |
| Prostate Cancer * | 100.0 | 100.0 | 1944.3 | 1951.2 |
| Breast Cancer * | 0.0 | 0.0 | NA | NA |
| Testicular Cancer * | 100.0 | 100.0 | 1953.2 | 1951.5 |

Out of Sample Quality Control

For out-of-sample testing, the inventors used the 2015 release of the Electronic Medical Records and Genomics (eMERGE) study of approximately 15k individuals available on dbGaP. The specific eMERGE data set used here refers to data downloaded from the dbGaP web site, under accession phs000360.v3.p1.(https://www.ncbi.nlm.nih.gov/projects/gap/cgi-bin/study.cgi?study_id=phs000360.v3.p1). The eMERGE dataset consists of 14,908 individuals with 561,490 SNPs which were genotyped on the Illumina Human 660W platform. The Plink 1.9 software is used for all further quality control. First, SNPs which are common to the UK Biobank can be filtered. SNPs and samples with missing call rates exceeding 3% are excluded and SNPs with minor allele frequency below 0.1% were also removed. This results in 557,595 SNPs and 14,906 individuals. Of these, the 468,514 SNPs which passed QC on the UK Biobank are used in training.

All eMERGE individuals in the dataset self-reported their ethnic ancestry as white. For purposes of the inventors' experiments, not all individuals in eMERGE were strictly cases or controls for any one particular condition. For Type 2 Diabetes, there were 1,921 identified cases and 4,369 identified controls. For Hypothyroidism there were 1084 identified cases and 3171 identified controls. For Hypertension, as the study focused on identifying individuals with Resistant Hypertension, there were two types of cases and two types of controls. Case group 1 consisted of subjects with 4 or more medications simultaneous on at least 2 occasions greater than one month apart. Case group 2 had two outpatient (if possible) measurements of systolic blood pressure over 140 or diastolic blood pressure greater than 90 at least one month after meeting medication criteria while still on 3 simultaneous classes of medication AND has three simultaneous medications on at least two occasions greater than one month apart. Control group 2 consists of subjects with no evidence of Hypertension. Control group 1 consisted of subjects with outpatient measurements of SBP over 140 or DBP over 90 prior to beginning medication AND has only one medication AND has SBP<135 and DBP<90 one month after beginning medication. For model testing of Hypertension, case group 1, case group 2 and control group 1 were classified as cases, while control group 2 is used as controls. For Resistant Hypertension, case group 1 and case group 2 were classified as cases, while control group 2 is used as controls—control group 1 is excluded from this testing. The size of the self-reported white members of the groups are: case group 1—952, case group 2—406, control group 1—677, control group 2—1202.

The year of birth in eMERGE is given by decade, so the year of birth is taken to be the 5th year of the decade (i.e., if the decade of birth is 1940, then 1945 is used as year of birth). Some individuals did not have a year of birth listed—these individuals are included when testing models which did not feature age and sex as covariates, but are excluded when testing a model which included age. For obtaining age and sex effects, the inventors used the entire UK Biobank for training as opposed to excluding younger participants as was done for the genetic models.

### Testing Using Genetically Dissimilar Subgroups: Ancestry Out-of-Sample Testing

For many case-control phenotypes, the inventors did not have access to a second data set for proper out-of-sample testing. For these traits, an ancestry out-of-sample (AOS) testing procedure was followed. In this procedure, the predictor is trained on individuals of a homogeneous ethnic background: from UKBB genetically British individuals are used, defined using principal components analysis of population data. The predictor is then applied to individuals who are genetically dissimilar to the training set but not overly distant. For the testing set self-reported white (i.e., European) individuals (British/Irish/Any Other White) who are not in the cohort identified as genetically British are used. These individuals might be, for example, people of primarily Italian, Spanish, French, German, Russian, or mixed European ancestry who now live in the UK.

To identify the genetically British individuals, the top 20 principal components for the entire sampled population are provided directly from UK Biobank and the top 6 are used to identify genetically British individuals. Individuals who self-report their ethnicity as “British” are selected, and the outlier detection algorithm from the R-package “Aberrant” is used to identify individuals using pairs of principal component vectors.

Aberrant uses a parameter which is the ratio of standard deviations of the outlying to normal individuals (λ) (Note λ here is a variable name used in Aberrant. It should not be confused with the lasso penalization parameter used in optimization). This parameter is tuned to make a training set which is overly homogenous compared to those reported as genetically British by the UKBB (λ˜20). Because Aberrant uses two inputs at a time, individuals to be excluded from training were identified using principal component pairs (first and second, third and fourth, fifth and sixth) and the union of these sets are the total group which is excluded in the final training set. There were a total of 402,937 individuals to be used in training after principal component filtering.

For this type of testing, the directly called gentoypes are used for training, cross-validation and testing (imputed SNPs are only used for true out-of-sample testing). First, only self-reported white individuals were selected (472,856) and then SNPs and samples with missing call rates exceeding 3% were removed, as were SNPs with minor allele frequency below 0.1% (all using Plink). This results in 658,543 SNPs and 459,039 total individuals which consists of 401,845 genetically British who are used for training and 57,194 non-British self-reported white individuals are used for final ancestry based out-of-sample testing.

### Odds Ratios for AOS

Here, the odds ratio cumulant plots were collected as a function of PGS percentile (i.e., a given value on the horizontal axis represents individuals with that PGS or higher) for the various phenotypes that were tested with the AOS procedure described above and reported in Table 1. Also, some comparisons to alternative methods for analyzing the genetic predictability of these phenotypes are commented on. It should be noted that some of these phenotypes—e.g., Asthma, Heart Attack, and High Cholesterol—have been heavily linked to other complex traits as well as external risk factors; thus, as additional data is added to a dataset and these additional traits and risk factors (e.g., smoking) become included in records in such datasets, predictor models generated from those datasets will greatly improve prediction.

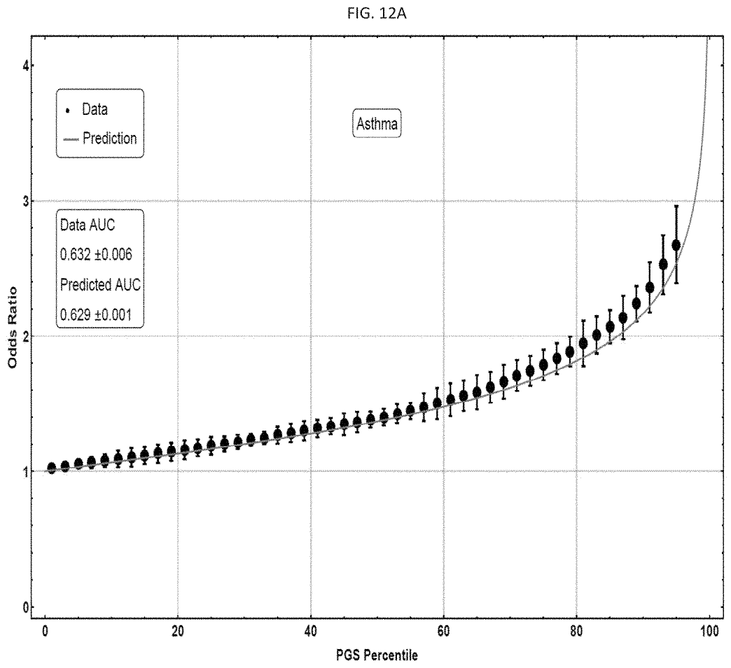

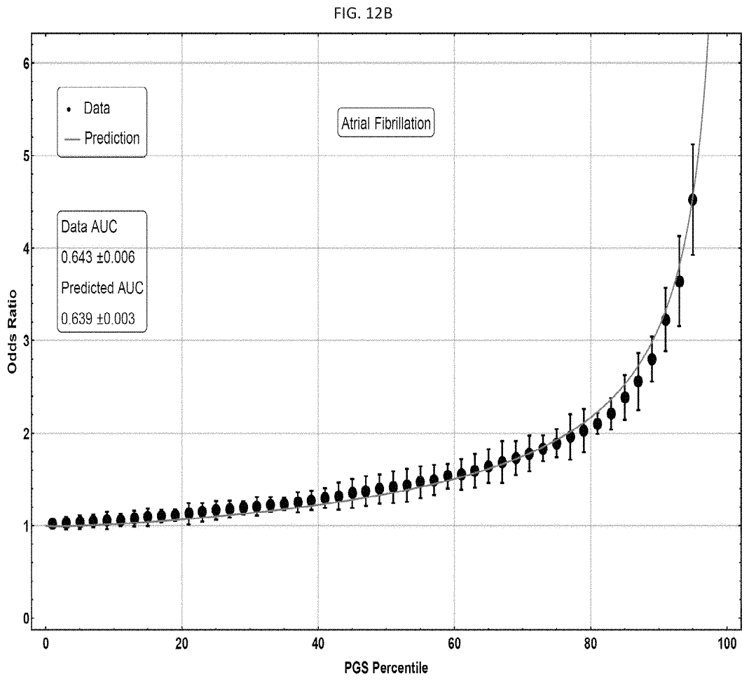

Asthma, in FIG. 12A, has long been known to have a significant genetic component. FIG. 12A is a graph of odds ratio as a function of PGS percentile (i.e. scores at that point or above) for Asthma. FIG. 12B is a graph of odds ratio as a function of PGS percentile for Atrial Fibrillation. In this study odds ratios ˜3× are found for people with PGS scores in the 96^{th} percentile and above. This compares favorably to the literature where 2.5× odds ratio increase at 95% confidence level was found for children with parents that have asthma. GWAS studies have shown that Asthma seems to be correlated with both hay fever and Eczema conditions. Although in performing this study, a strong predictor for Eczema was not found, relevant data is available in UKBB and multi-phenotype studies could be performed in the future. For example, in alternative embodiments, a priori knowledge of the association of two conditions, such as Eczema and Asthma could be utilized in one of two ways to reveal a stronger predictor for Eczema without having to obtain massive datasets (which may not be readily available). In one embodiment, a predictor for Asthma may be developed indicating an individual has a high likelihood for developing Asthma. That likelihood of Asthma could be utilized as a phenotypic datapoint (e.g., “Asthma Likely”) that can be added to a regression for a potential Eczema predictor. Thus, a stronger predictor for Eczema could at least be found for patients who already have a risk of Asthma. In another embodiment, Asthma and Eczema (or other highly correlated disease conditions) could be combined in a multi-phenotype study in which the “cases” are individuals who have both Asthma and Eczema.

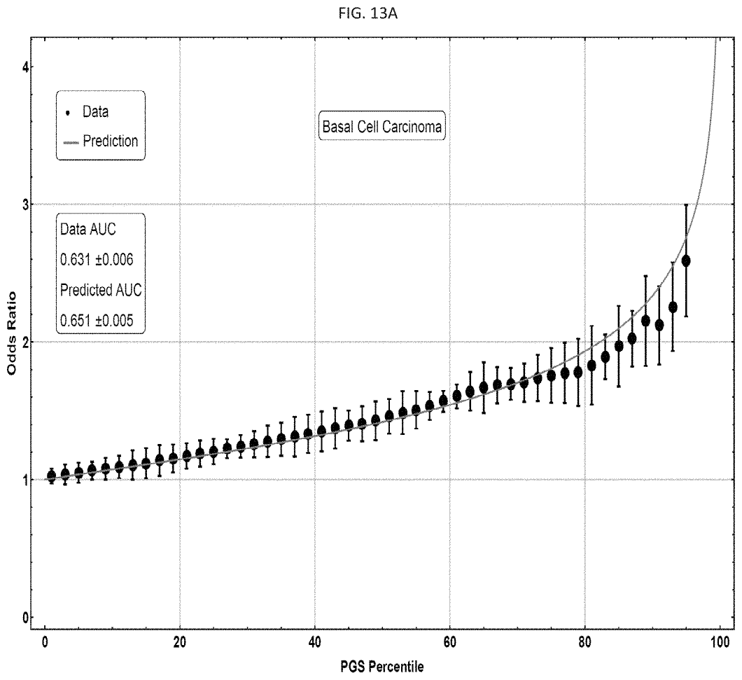

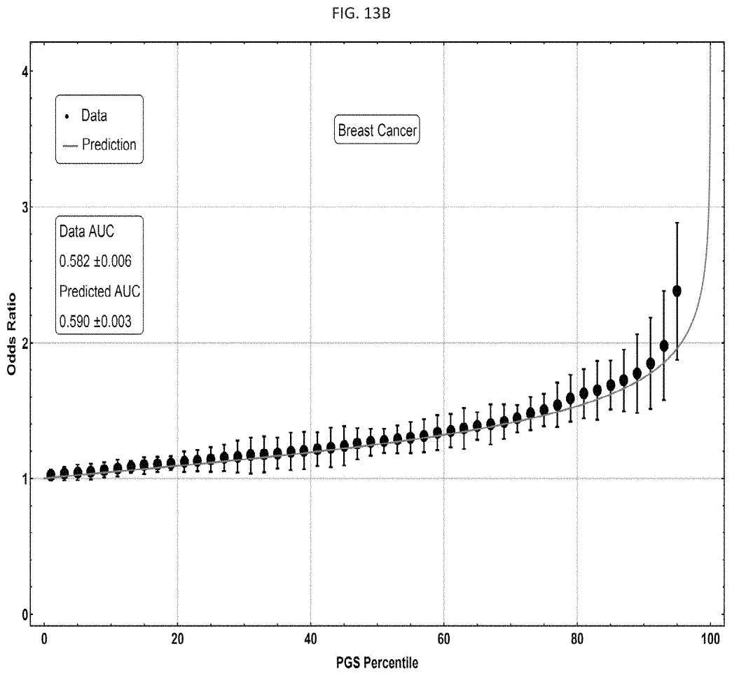

Atrial Fibrillation, seen in FIG. 12B, is also known to have a genetic risk factor. Parental studies have shown a 1.4× odds ratio, but-although gene loci have been identified, genetic studies have not previously been successful in clinical settings. In this work, PGS scores in the 96^{th} percentile and above predict up to a 5× increase in odds. FIG. 13A is a graph of odds ratio as a function of PGS percentile (i.e. scores at that point or above) for Basal Cell Carcinoma. FIG. 13B is a graph of odds ratio as a function of PGS percentile for Breast cancer. Breast Cancer, in FIG. 13B, has long been evaluated with the understanding that there is a genetic risk component. Recent studies involving multi SNP prediction (77 SNPs) have been able to predict 3× odds increases for genetic outliers. This is consistent with the results for the highest genetic outliers although many more SNPS 480±62 were used by the inventors.

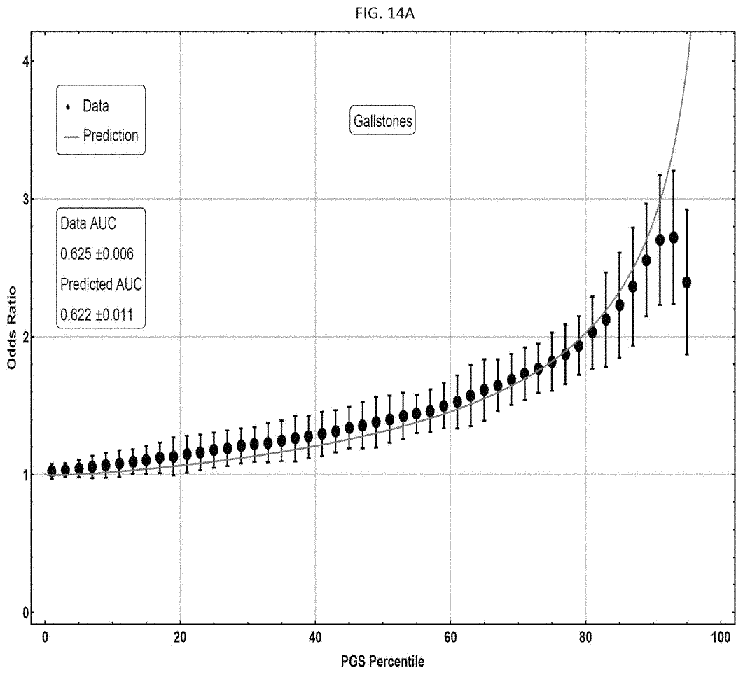

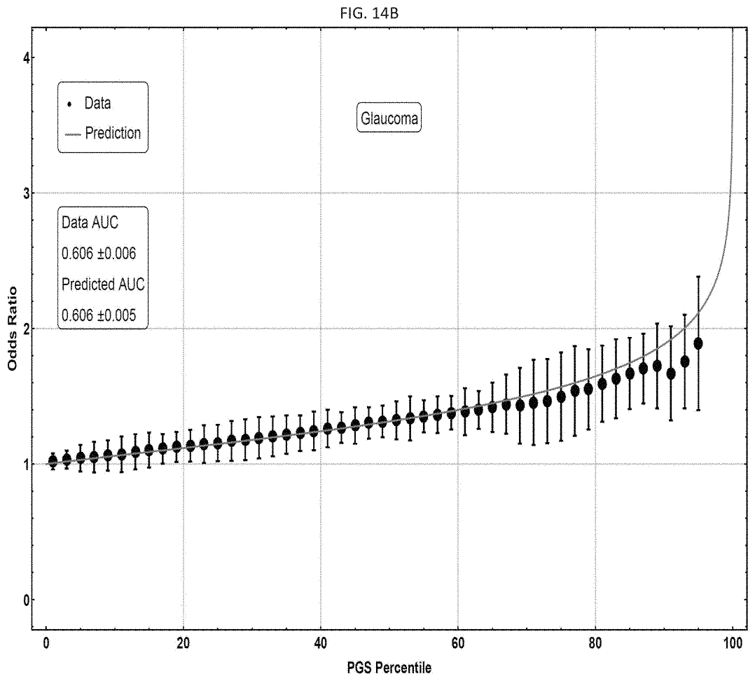

Recent reviews suggest that much of the risk leading to a higher probability of having Gallstones is associated with non-genetic factors. However, in FIG. 14, the inventors found that 90^{th} percentile and above PGS is be associated with a 3× odds increase. FIG. 14A is a graph of odds ratio as a function of PGS percentile (i.e. scores at that point or above) for Gallstones. FIG. 14B is a graph of odds ratio as a function of PGS percentile for Gluacoma.

While there are a variety of relevant environmental factors, recent reviews of the genetics of Glaucoma highlight that GWAS studies have found 25 genic regions with odds ratios above 1×, the highest being 2.80×. In FIG. 14B similar odds ratios for extreme PGS are seen.

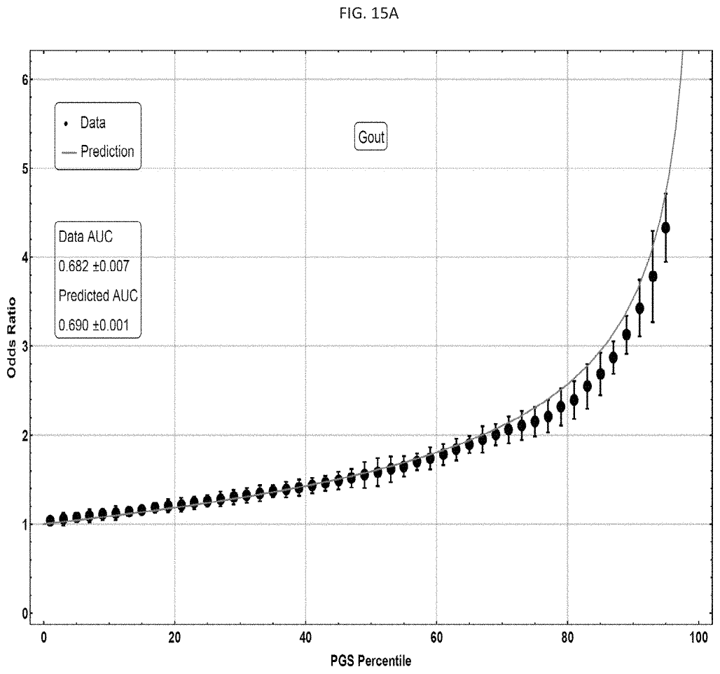

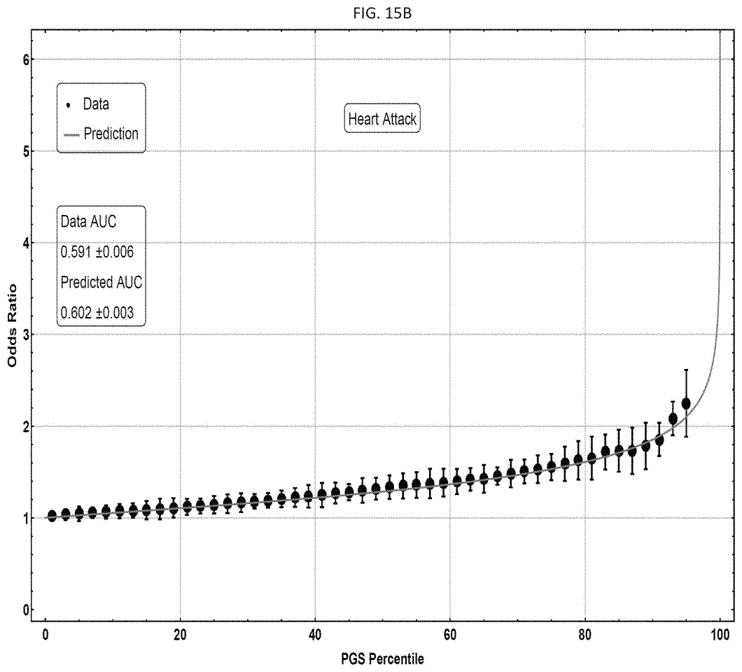

Gout, seen in FIG. 15A, has an extremely high 4.5× odds ratio for PGS in the 96^{th} percentile and above. Reviews of Gout have noted both a strong familial heritability and known GWAS loci, but the inventors are not aware of previously-computed odds ratios this large solely due to genetics. FIG. 15A is a graph of odds ratio as a function of PGS percentile (i.e. scores at that point or above) for Gout. FIG. 15B is a graph of odds ratio as a function of PGS percentile for Heart Attack.

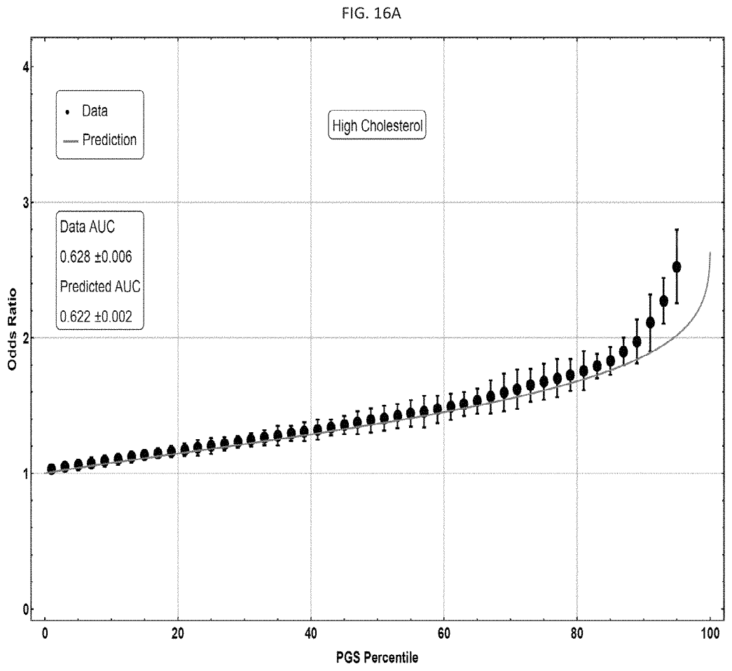

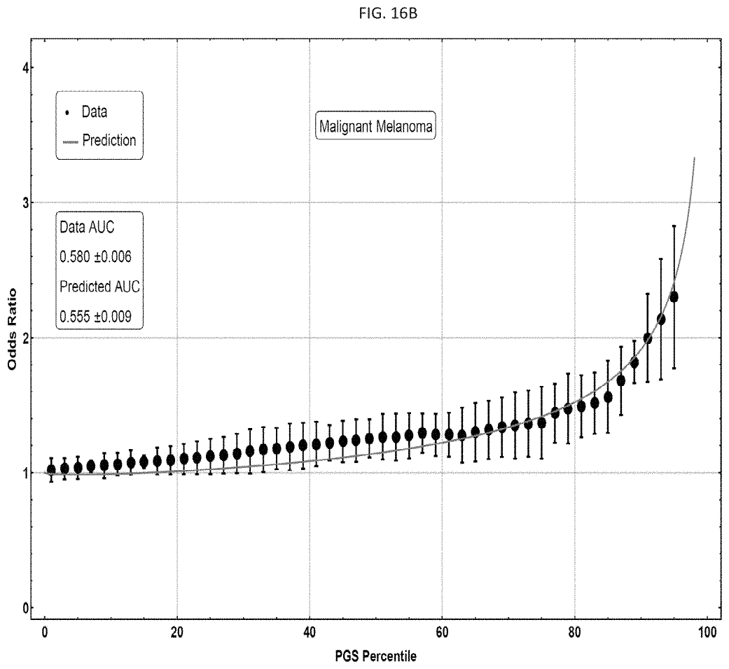

FIG. 16A is a graph of odds ratio as a function of PGS percentile (i.e. scores at that point or above) for High Cholesterol. FIG. 16B is a graph of odds ratio as a function of PGS percentile for Malignant Melanoma.

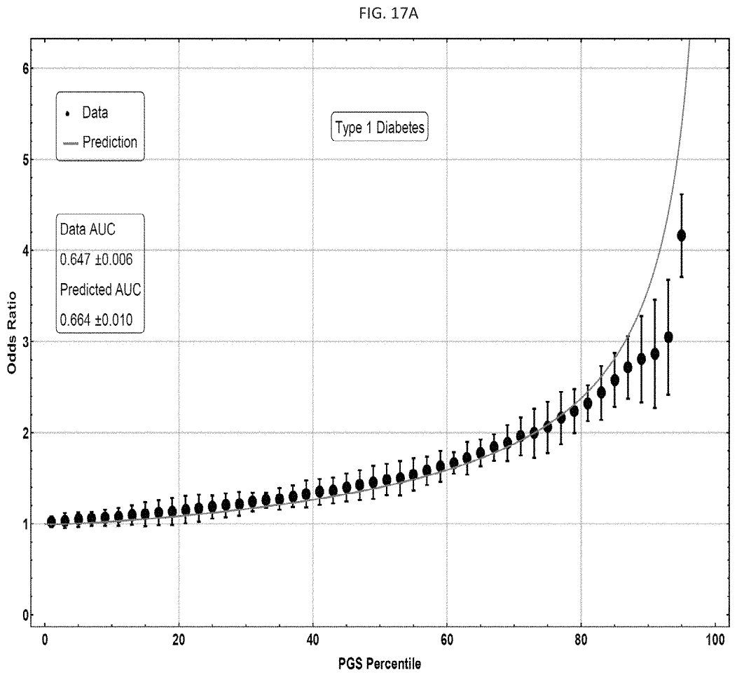

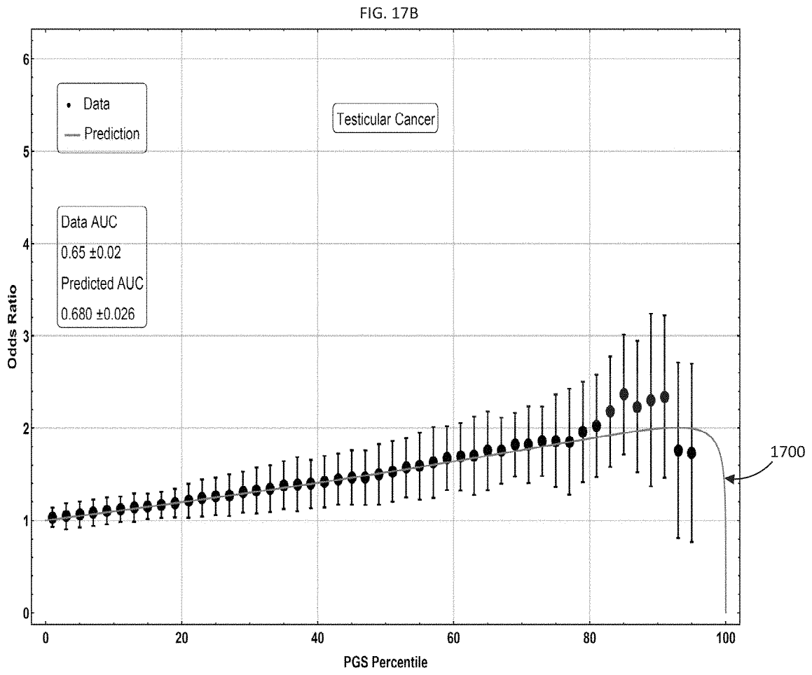

There is a wide ranging literature covering the genetics and heritability of Type 1 Diabetes. In FIG. 17A a large 4.5× odds ratio is seen for extreme PGS. Notably, the literature has identified genetic prediction to be extremely useful in differentiating between Type 1 and Type 2 Diabetes and in identifying β cell autoimmunity, which is highly correlated with diabetes. FIG. 17A is a graph of odds ratio as a function of PGS percentile (i.e. scores at that point or above) for Type 1 Diabetes. FIG. 17B is a graph of odds ratio as a function of PGS percentile for Testicular Cancer. Note that the dip at extreme PGS values in a predicted Testicular Cancer curve 1700 may be related to a small number of available cases; the cases and controls are not well fit by two separate Gaussian distributions.

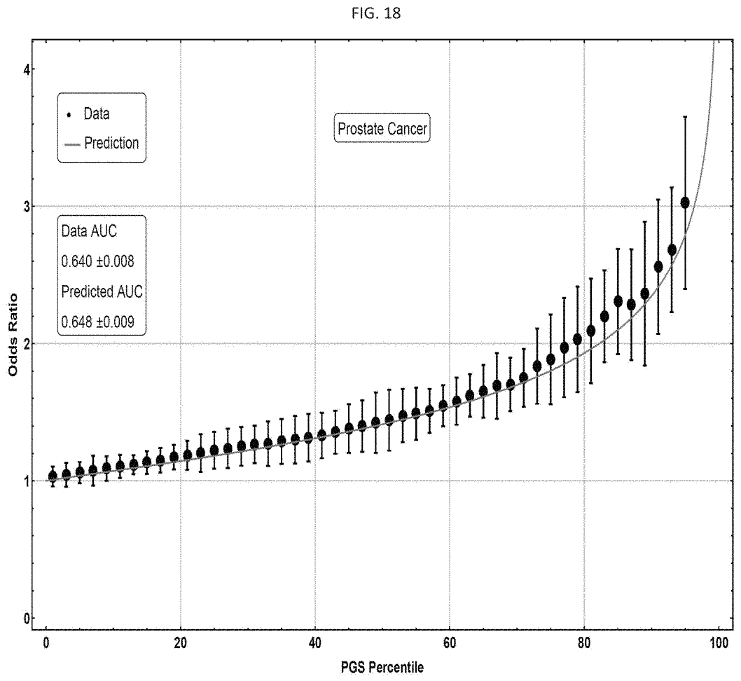

Prostate Cancer is the most common gender specific cancer in men. FIG. 18 is a graph of odds ratio as a function of PGS percentile (i.e. scores at that point or above) for Prostate Cancer. It has long been known that age is a significant risk factor for prostate cancer, but GWAS studies have shown that there is a significant genetic component. Additionally, it has been shown, using genome wide complex trait analysis (GCTA), that variants with minor allele frequency 0.1-1% make up an important contribution to “missing heritablity” for men of African ancestry. This study includes some SNP variants with minor allele frequency as low as 0.1%, so the model might include some of this contribution.

### Model Training Algorithm

In some embodiments, the generation of a predictor model for a given trait or disease condition can entail a custom implementation of a LASSO regression (Least Absolute Shrinkage and Selection Operator). Other alternative methods may include the use of machine learning techniques such as gradient boosted trees, random forest, kth nearest neighbors, and the like. For the inventors' experiments however, it was found that custom regression techniques provided the best output. However, it should be kept in mind that a system that utilizes predictive models to provide risk scores to a user need not operate in an either/or realm. For example, as datasets become more robust (e.g., including more records, more SNPs, and/or more phenotypic data), it may be that certain machine learning techniques begin to achieve superior results. At that point a deep learning-trained model could be substituted in place of, or combined with, a predictive model generated by custom LASSO for a given trait.

The inventors prepared custom LASSO implementations using the Julia language, although other programming languages are also possible for use. Given a set of samples i=1, 2, . . . , n with a set of p SNPs, the phenotype yᵢ and state of the j^{th} SNP, Xᵢⱼ, are observed. Xᵢⱼ is an n×p matrix which contains the number of copies of the minor allele and any missing values are replaced with the SNP average. The L₁ penalized regression, LASSO, seeks to minimize the objective function

O_{λ}(\vec{β})=½∥\vec{y}−X\vec{β}∥²+nλ∥\vec{β}∥₁ (E.1)

where |\vec{v}|=Σᵢⁿ|vᵢ| is the L₁ norm, ∥\vec{v}∥=Σᵢⁿvᵢ² is the L₂ norm and λ is a tunable hyperparameter. The solution is given in terms of the soft-thresholding function as

$$S ( z , γ ) = sgn ( z ) max ( \vert z \vert - γ , 0 ) \\ {β}_{j}^{*} = \frac{1}{{\sum }_{i = 1}^{n} {X}_{ij}^{2}} S ( {\sum }_{i = 1}^{n} [ {X}_{ij} {y}_{i} - {\sum }_{k \neq j} {X}_{ij} {X}_{ik} {β}_{k} ] , n λ ) \qquad ( E .2 )$$

The penalty term affects which elements of \vec{B} have non-zero entries. The value of λ is first chosen to be the maximum value such that all βᵢ are zero, and it is then decreased, allowing more nonzero components in the predictor. For each value of λ, \vec{β}*(λₙ) is obtained using the previous values of \vec{β}* (λₙ₋₁) (warm start) and coordinate descent. The Donoho-Tanner phase transition describes how much data is required to recover the true nonzero components of the linear model and suggests that the true signal can be recovered with s SNPs when the number of samples is n˜30s-100s (see [45, 50]).

For all three conditions which are available in eMERGE, a subset of 1000 cases and 1000 controls was withheld from the training set to be set aside for cross-validation. This process was repeated 5 times with non-overlapping cross-validation sets. With training and cross-validation sets constructed, a GWAS is performed on the training set and select the rank ordered top 50,000 p-value SNPs. Then these SNPs are used as input to the LASSO algorithm and finally apply the predictor to the corresponding cross-validation set in order to select the value of λ. For conditions which AOS testing is used, cross-validation sets of 500 cases and 500 controls were used to tune the model.

Because individual SNPs are uncorrelated to year of birth and sex, SNPs can be regressed on independently of age and sex. To train combined models, which include SNPs, age, and/or sex, LASSO can be performed on SNPs alone and least squares regression on age and sex only, then add the two predictor scores together. The inventors tested for whether an improvement in AUC is achieved through a simultaneous regression using polygenic score (PGS), age, and sex as covariates, but found this to give similar AUC as doing the regressions independently and adding the results (to within a few % accuracy). However, it is contemplated that datasets of increased size and depth of detail per record may benefit from combining SNPs, age, sex, and/or other non-genomic attributes.

### Analytic AUC and Risk

As described above, risk score functions can be determined from analysis of AUC. By assuming that cases and controls within a given dataset have PGS distribution which is Gaussian, quantities can be analytically calculated for genetic prediction. For example, an AUC can be calculated and analyzed to see how it corresponds to an odds ratio for various distributional parameters.

Assume a case-control phenotype and that the cases and controls have Gaussian distributed PGS. Letting i={0,1} represent controls and cases respectively, the distribution of scores can be written

$$f ( x ) = \frac{1}{{n}_{0} + {n}_{1}} {\sum }_{i = 0 , 1} {n}_{i} {f}_{i} ( x ) \\ f ( x , {μ}_{i} , {σ}_{i} ) ≡ {f}_{i} ( x ) = \frac{1}{\sqrt{2 π}} Exp ( \frac{- {( x - {μ}_{i} )}^{2}}{2 {σ}_{i}^{2}} ) , \qquad ( F .1 )$$

and nᵢ represents the total number of cases/controls. For completeness, the definition of the error function is recalled here

$$Erf ( x ) ≡ \frac{2}{\sqrt{π}} {\int }_{0}^{x} {e}^{- {t}^{2}} dt .$$

AUC

To calculate AUC, an ROC curve of false positive rate (FPR) vs true positive rate (TPR) is first generated.

$$\begin{matrix}FPR ( z , {μ}_{0} , {σ}_{0} ) ≡ \frac{false positives}{false positives + true negatives} = \frac{{\int }_{z}^{∞} {n}_{0} {f}_{0} ( x ) dx}{{\int }_{z}^{∞} {n}_{0} {f}_{0} ( x ) dx + {\int }_{- ∞}^{z} {n}_{0} {f}_{0} ( x ) dx} & ( F .2 ) \\ = {\int }_{z}^{∞} \frac{1}{\sqrt{2 π}} Exp ( \frac{- {( x - {μ}_{0} )}^{2}}{2 {σ}_{0}} ) dx = \frac{1}{2} ( 1 - Erf ( \frac{z - {μ}_{0}}{\sqrt{2} {σ}_{0}} ) ) & ( F .3 ) \\ = 1 - Φ ( \frac{z - {μ}_{0}}{{σ}_{0}} ) & ( F .4 ) \\ TPR ( z , {μ}_{1} , {σ}_{1} ) ≡ \frac{true positives}{true positives + false negatives} = \frac{1}{2} ( 1 - Erf ( \frac{z - {μ}_{1}}{\sqrt{2} {σ}_{1}} ) ) & ( F .5 ) \\ = 1 - Φ ( \frac{z - {μ}_{1}}{{σ}_{1}} ) . & ( F .6 )\end{matrix}$$

The AUC is then defined as the area under the ROC curve,

$$\begin{matrix}\begin{matrix}AUC ( {μ}_{0} , {σ}_{0} , {μ}_{1} , {σ}_{1} ) = {\int }_{- ∞}^{∞} TPR ( FPR ( z , {μ}_{0} , {σ}_{0} ) , {μ}_{1} , {σ}_{1} ) dz \\ = {\int }_{- ∞}^{∞} TPR ( z , {μ}_{1} , {σ}_{1} ) {\partial }_{z} FPR ( z , {μ}_{0} , {σ}_{0} ) dz\end{matrix} & ( F .7 ) \\ = {\int }_{- ∞}^{∞} \frac{1}{2} ( 1 - Erf ( \frac{z - {μ}_{1}}{\sqrt{2} {σ}_{1}} ) ) ( \frac{Exp ( \frac{- {( z - {μ}_{0} )}^{2}}{2 {σ}_{0}^{2}} )}{\sqrt{2 π} {σ}_{0}} ) dz & ( F .8 ) \\ = \frac{1}{2} - \frac{{σ}_{1}}{2 \sqrt{π} {σ}_{0}} {\int }_{- ∞}^{∞} Erf ( y ) Exp ( - {( \frac{{σ}_{1}}{{σ}_{0}} y + \frac{{μ}_{1} - {μ}_{0}}{\sqrt{2} {σ}_{0}} )}^{2} ) dy & ( F .9 ) \\ = \frac{1}{2} ( 1 + Erf ( \frac{{μ}_{1} - {μ}_{0}}{\sqrt{2 ( {σ}_{1}^{2} + {σ}_{0}^{2} )}} ) ) = Φ ( \frac{{μ}_{1} - {μ}_{0}}{\sqrt{( {σ}_{1}^{2} + {σ}_{0}^{2} )}} ) , & ( F .10 )\end{matrix}$$

in agreement with Eq.(3.1). Note that the AUC is independent of the number of cases and controls.

Risk and Odds

Next, the increased likelihood of a disease at a higher z-score is classified. As disclosed herein, at least two alternate methods could be employed.

Risk Ratio represents the ratio between (a) the number of cases at a particular z-score and above over the total number of people at z-score and above to (b) the total number of cases over the total number of cases and controls.

$$\begin{matrix}RR ( {μ}_{0} , {σ}_{0} , {μ}_{1} , {σ}_{1} , {n}_{0} , {n}_{1} ) = \frac{( {\int }_{z}^{∞} {n}_{1} {f}_{1} ( x ) dx ) / ( {\int }_{z}^{∞} ( {n}_{1} {f}_{1} ( x ) + {n}_{0} {f}_{0} ( x ) ) dx )}{{n}_{1} / ( {n}_{0} + {n}_{1} )} & ( F .11 ) \\ RR ( {μ}_{0} , {σ}_{0} , {μ}_{1} , {σ}_{1} , r ) = ( \frac{1}{r} + 1 ) {( 1 + \frac{1 - Erf ( \frac{z - {μ}_{0}}{\sqrt{2} {σ}_{0}} )}{1 - Erf ( \frac{z - {μ}_{1}}{\sqrt{2} {σ}_{1}} )} )}^{- 1} & ( F .12 ) \\ = ( \frac{1}{r} + 1 ) {( 1 + \frac{1 - Φ ( \frac{z - {μ}_{0}}{{σ}_{0}} )}{1 - Φ ( \frac{z - {μ}_{1}}{{σ}_{1}} )} )}^{- 1} , & ( F .13 )\end{matrix}$$

where it is noted that the Risk Ratio only depends on the ratio r≡n₁/n₀.

Odds Ratio represents the ratio between (a) the number of cases at a particular z-score and above over the number of controls at a particular z-score and above to (b) the total number of cases over the total number of controls

$$\begin{matrix}OR ( {μ}_{0} , {σ}_{0} , {μ}_{1} , {σ}_{1} , {n}_{0} , {n}_{1} ) = \frac{( {\int }_{z}^{∞} {n}_{1} {f}_{1} ( x ) dx ) / ( {\int }_{z}^{∞} {n}_{0} {f}_{0} ( x ) dx )}{{n}_{1} / {n}_{0}} & ( F .14 ) \\ OR ( {μ}_{0} , {σ}_{0} , {μ}_{1} , {σ}_{1} ) = \frac{1 - Erf ( \frac{z - {μ}_{1}}{\sqrt{2} {σ}_{1 }} )}{1 - Erf ( \frac{z - {μ}_{0}}{\sqrt{2} {σ}_{0}} )} = \frac{1 - Φ ( \frac{z - {μ}_{1}}{{σ}_{1}} )}{1 - Φ ( \frac{z - {μ}_{0}}{{σ}_{0}} )} , & ( F .15 )\end{matrix}$$

which is independent of n₁ and n₀. This is the result Eq.(3.2). Note that in the rare disease limit (RDL)

$${n}_{1} << {n}_{0} and \\ {n}_{1} ( 1 - Erf ( \frac{z - {μ}_{1}}{\sqrt{2} {σ}_{1}} ) ) << {n}_{0} ( 1 - Erf ( \frac{z - {μ}_{0}}{\sqrt{2} {σ}_{0}} ) ) , \qquad ( F .16 )$$

the risk ratio and odds ratio agree

$$RR ( {μ}_{0} , {σ}_{0} , {μ}_{1} , {σ}_{1} , r ) {}^{RDL} OR ( {μ}_{0} , {σ}_{0} , {μ}_{1} , {σ}_{1} ) . \qquad ( F .17 )$$

PGS percentile: In either case, it is of interest to know the risk or odds ratio in terms of the percentage of people with a particular z-score and above. The percentile function can be defined as

$$P ( z , {μ}_{0} , {σ}_{0} , {n}_{0} , {μ}_{1} , {σ}_{1} , {n}_{1} ) = \frac{1}{{n}_{0} + {n}_{1}} {\int }_{∞}^{z} ( {n}_{0} {f}_{0} ( x ) + {n}_{1} {f}_{1} ( x ) ) dx = \frac{1}{2 ( 1 + r )} ( 1 + Erf ( \frac{z - {μ}_{0}}{\sqrt{2} {σ}_{0}} ) + r ( 1 + Erf ( \frac{z - {μ}_{1}}{\sqrt{2} {σ}_{1}} ) ) ) = \frac{1}{1 + r} ( Φ ( \frac{z - {μ}_{0}}{{σ}_{0}} ) + r Φ ( \frac{z - {μ}_{1}}{{σ}_{1}} ) ) = P ( z , {μ}_{0} , {σ}_{0} , {μ}_{1} , {σ}_{1} , r ) . \qquad ( F .18 )$$

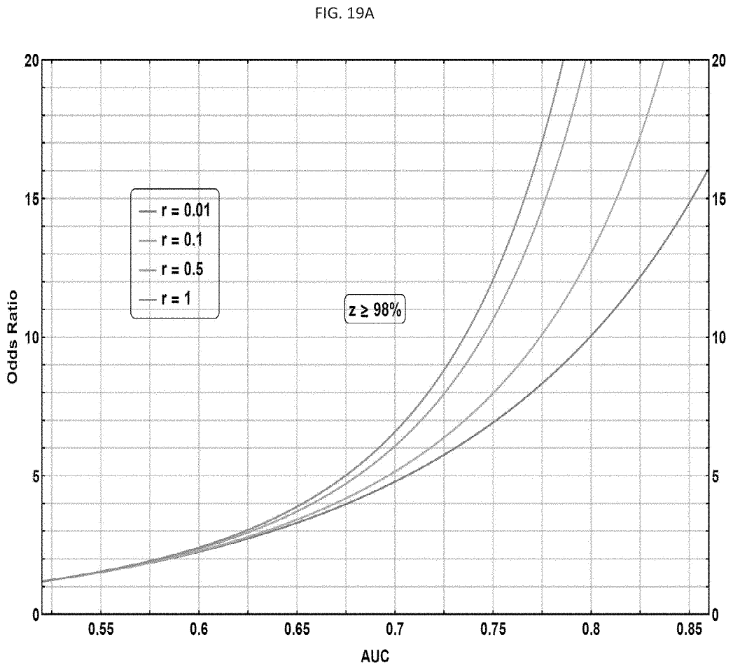

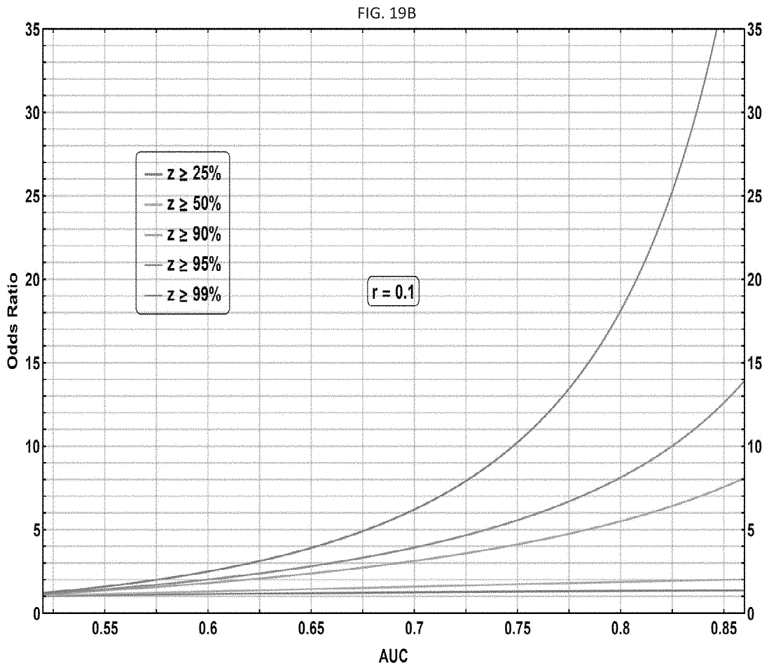

Combining Eq.(3.1), Eq.(3.2), and Eq.(F.18) the odds ratio can be plotted in terms of the distributional parameters as seen in FIG. 19. Odds ratio (assuming two displaced Gaussian distributions) as a function of AUC. FIG. 19A is a graph of the odds ratio as a function of AUC for z-scores above the 98^{th} percentile at various values of the ratio of cases to controls r. FIG. 19B is a graph of the odds ratio as a function of AUC for case to control ratio r=0.1 at various z-score percentiles. Assuming a population-representative sample, r is the prevalence of the disease in the general population.

### Implementations and Embodiments

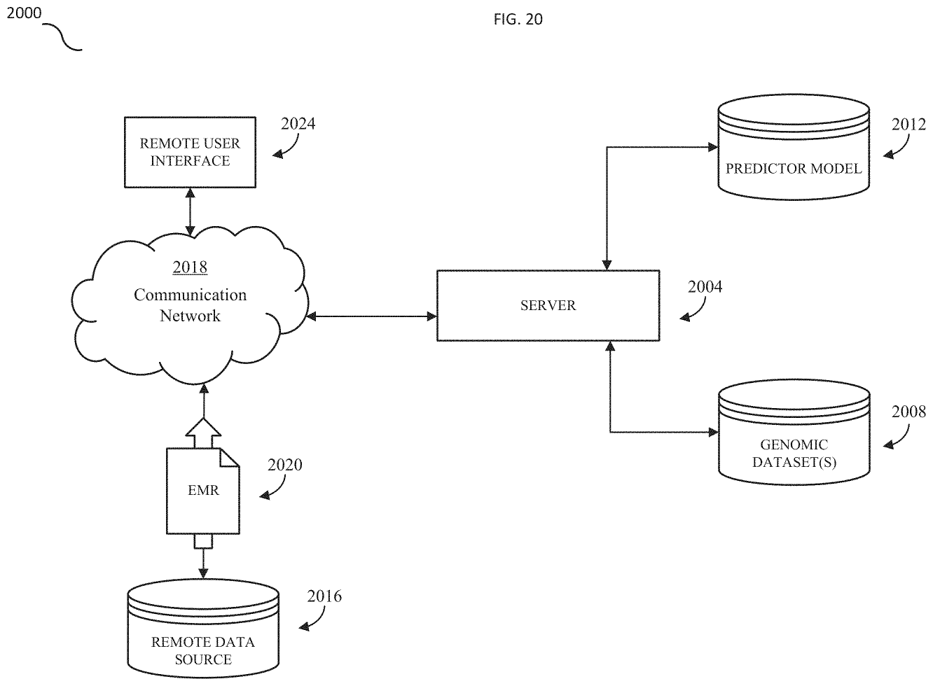

Referring now to FIG. 20, an exemplary system 2000 for distributing and processing genomic data and predicting polygenic disease risk scores is shown. The system can include a risk score processing server 2004 including one or more processors. In practice, this server could be simply virtual, or it could be an integration within a electronic medical records server. But regardless of implementation, server 2004 would be running an application that causes it to operate to receive user requests for risk scores, input genomic and other data relating to those user requests, pre-process the data, then generate one or more polygenic risk scores and return them to the user. The server 2004 can be coupled to and in communication with one or more memories. In some embodiments, the server 2004 can be coupled to and in communication with a first memory 2008 and a second memory 2012, each of which could be virtual cloud storage space, or local drives (which in some circumstances may be preferable for purposes of data privacy). The first memory 2008 can include one or more genomic datasets, such as the UKBB or eMERGE datasets, or a non-public dataset, or any combination of such datasets. The datasets in the first memory are stored for purposes of generation of predictor models according to the methods and techniques described above. Thus, this dataset need not be accessed on a regular basis by server 2004, and can be anonymized and pre-processed into a uniform format. In some embodiments, new user data received by server 2004 for purposes of providing a risk score could then be formatted and anonymized and added to the datasets stored thereon. The second memory 2012 can include one or more predictor models, which can be generated using the techniques described above. The predictor models of memory 2012 corresponding to a particular user request are then accessed by the server 2004 as it calculates a risk score profile to return to the user. Similarly, over time the predictor models of memory 2012 may be updated based on further training data available in memory 2008. Each predictor model is tagged with a corresponding set of use case data, indicating which types of user requests it would be most appropriate for. In some embodiments, the server 2004 can be in communication with a single memory including the datasets and the predictor model.

The server 2004 can be in communication with a remote data source 2016 via a communication network 2018. The communication network 2018 can include an Internet connection, a LAN connection, a healthcare records infrastructure, or other similar connections. The remote data source 2016 could be, for example a server of a genotyping lab company, a healthcare institution that can provide access to one or more electronic medical records (EMRs) 2020 to the server 2004, an insurer, or even simply an individual user logged into a web-based portal. The server 2004 can be in communication with a remote user interface 2024 that can be included in a smartphone, computer, tablet, display screen, etc. In some embodiments, the server 2004 can be in communication with multiple user interfaces, each user interface being associated with a patient or medical practitioner. In other embodiments, the server 2004 could be in communication with simply a plug-in of a healthcare records network, such as an electronic medical records platform.

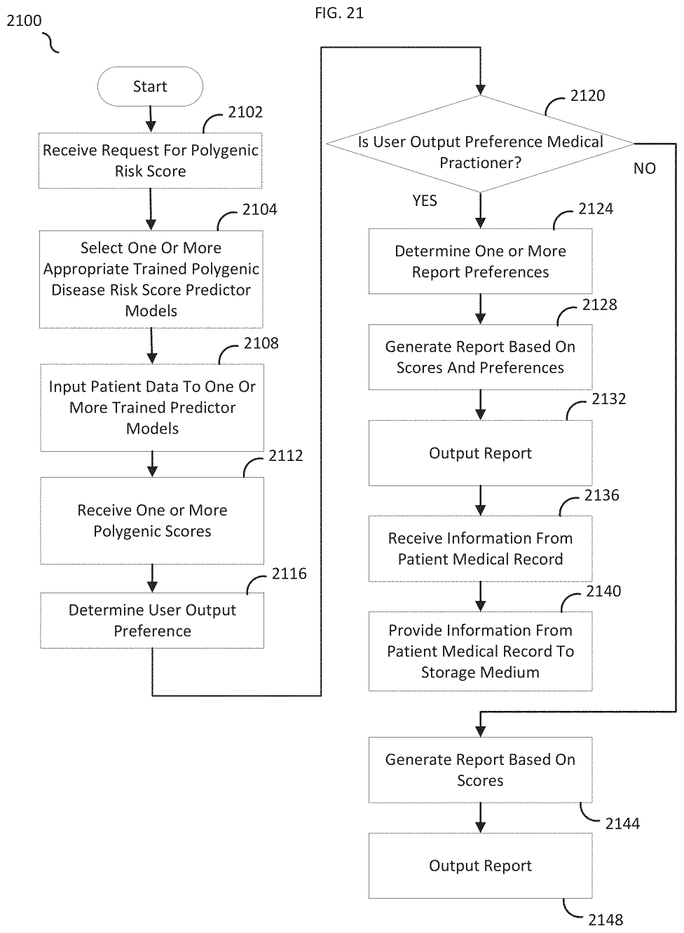

Referring now to FIG. 20 as well as FIG. 21, an exemplary process 2100 for providing a polygenic disease risk score based on genomic data is shown. This process could be implemented through, for example, a system architecture as shown in FIG. 20. The process can provide a user (whether an individual, a physician, a genetic counselor, an insurer, or other user) with a polygenic risk score (such as a broad disease risk screen, a specific targeted prediction of certain phenotypic characteristics, or some combination thereof) based on an individual's genomic data (alone or in combination with other information such as age and sex of the individual).

At step 2102, the process 2100 can begin upon receipt of a request for a polygenic risk score. This request would be received from a user, either remote or part of the same network as the server running the process 2100. The request could contain patient data, or may simply provide the appropriate permissions and direct the server to acquire patient data from another resource (e.g., an EMR or a genotyping lab). The patient data can generally be associated with a single patient. The patient data can include all or a selected portion of the result of a genotyping analysis of the patient, and other data concerning the patient such as an age value, a sex value, a self-reporting of ethnicity, medical condition information as described above, and/or various individual or time series health testing data (e.g., blood pressure measurements, weight measurements, etc.). In one embodiment, this data may be entered into a user interface by the patient or another user. In another embodiment, this data and the associated request may be automatically generated by a health care record upon the occurrence of some event (e.g., a battery of tests upon a patient being admitted into a hospital, or a periodic physical, or an application for life insurance). The process 2100 can then proceed to 2104.

At 2104, the process 2100 can select one or more appropriate trained polygenic disease risk score predictor models for the patient based on a number of factors. First, the user request received in step 2102 can dictate to some extent which group of predictor models should be considered for the given patient—if the patient only requested a prediction of height, heart disease, or another individual or narrow category of traits, then predictor models for other traits need not be considered. In other embodiments, a preset or default set of predictions can be made for every request in addition to or instead of a user's request. For example, if an individual's age, weight, and gender put them in an epidemiologically-determined risk group for heart disease, the system might override the user's request and determine risk scores at least for cardiovascular diseases (in addition to any other traits the user had requested). Or, some healthcare providers or insurers may have preset default predictors they have requested for all of their patients, which can be stored as automatic settings for requests from those institutions.

Once the categories of disease conditions or phenotypic traits that should be analyzed for a given user request has been determined, in some system implementations the process can simply select the corresponding predictor model(s) for those conditions or traits. In other embodiments, however, there may be multiple predictor models for a given disease condition or trait that have been tuned to particular circumstances of a patient's background. For example, it may be that different predictive models are used based on a patient's ancestry or ethnicity. Or, a given predictive model for a certain disease state might be more accurate when taking into account the sex chromosomes, or different predictive models may provide better accuracy when age and sex are included in the training set—but a different predictive model is needed when age and sex data for a given patient are not available. In other circumstances, if certain SNP information is missing from the patient's genotype data, then it may be possible to simply use a less refined predictor that does not rely on the missing NSP information. The process 2100 can then proceed to 2108.

At 2108, the process 2100 then inputs the age value, the sex value, and the genotype data associated with the requesting patient to each of the one or more polygenic disease risk score predictor models selected at 2104. In some circumstances, the process will cull SNPs from the genotype data so that there is a correspondence between the SNPs presented to the predictor model and the SNPs the predictor model analyzes. The process 2100 can then proceed to 2112.

At 2112, the process 2100 receives a polygenic score from each of the one or more predictor models. As described above, each polygenic score can indicate a predicted risk level of a given disease or trait for the patient. For example, the process 2100 can receive a predicted height value from a height predictor model. The predicted height value can be an estimated height of the patient when fully grown. The predicted height value can be especially valuable if the patient is a child or adolescent as will be explained below. The process 2100 can then validate that the results present valid information and proceed to 2116.

At 2116, the process 2100 can determine a user output preference indicating who the polygenic disease risk scores will be compiled for and in what manner. For example, a user output preference can indicate the predictor output is intended for a physician or other medical practitioner, a patient, an EMR, a business (e.g., insurer), or other recipient. If, for example, the intended recipient is a medical practitioner's office or an EMR, the process 2100 may generate a report including full results. If the report is intended for a private individual, the report may be culled so that only predictions having a given significance are presented, or further explanations of predictions can be provided to help a layperson better understand the results. In one embodiment, an insurer may merely be notified that the predictions were generated and sent to a patient's physician, but actual risk scores are not provided to the insurer to protect patient privacy. The process 2100 can then proceed to 2120.

At 2120, if the user output preference is a medical practitioner (e.g., “YES” at 2120), the process 2100 can proceed to 2124. If the user output preference is a patient (e.g., “NO” at 2120), the process 2100 can proceed to 2144.

At 2124, the process 2100 can determine one or more report preferences from the medical practitioner. The medical practitioner can select report preferences using a dashboard provided on the remote display accessed by a web-based portal. The report preferences can include a threshold of likelihood value for each disease. For example, the threshold of likelihood can be twice the average chance of the disease in a population associated with the polygenic disease risk score predictor models (i.e., the British population). The report preferences can be used to only include disease risk scores that are significantly higher than average for a given population (e.g., in a specific geographic region, age group, etc.) in a report. In some embodiments, the report can compare the polygenic risk scores to epidemiologically-determined risk factors based on data such as a blood pressure readings, height, weight, age, etc. of the patient. In some embodiments, the process 2100 can compare a predicted height value of the patient at the patient's given age, to the current height of the patient to determine if the patient is on track to reach the predicted height value. The process 2100 can determine what percentile of adult heights the predicted height would fall into and what percentile the current measured height of the patient fall into compared to other patients in the same age group using reference data (e.g., a database of heights for given ages). If the percentile that the predicted height falls into is abnormally different than the percentile the current measured height falls into (e.g. more than one standard deviation away), the process 2100 can include a warning that the patient may not be growing properly in the report. In this way, the physician may be able to better decide if a child patient is not growing properly or if the child patient may be naturally short, and is therefore growing properly. For example, in some instances where a predictor indicates a child's adult height would be unusually low (or would be within a range which, at the low end, would indicate unusual lack of height), the process may suggest certain interventions to a physician. These suggestions could be automated, or could be upon user request. In one embodiment, a suite of predictors may always be run on each patient data set regardless of what prompted the patient or physician to request a polygenic analysis. One of the automatic predictors may be a height predictor if the patient is a child or adolescent. In that embodiment, the process could, unprompted, flag to a physician that certain interventions may be advisable. For example, if a height predictor indicates an abnormality in the child's current growth rate or predicted final height, the system could suggest to the physician that a certain regimen of growth hormone treatment should be considered or a diet change or other intervention be prescribed. The process 2100 can then proceed to 2128.

At 2128, the process 2100 can generate a report based on one or more received polygenic disease risk scores and the report preferences. The report can include the actual polygenic disease risk scores (e.g., “raw data”), and/or charts, graphs, and/or other visual aids generated based on the polygenic disease risk scores. The process 2100 can filter out any polygenic disease risk scores that are below the threshold of likelihood value set by the medical practitioner. The process 2100 can then proceed to 2132.

At 2132, the process 2100 can output the report to at least one of a display and a memory for storage. The display can be a remote user interface such as the remote user interface 2024, and can be located in view of a medical practitioner such as a physician who may use the report and/or the polygenic disease risk score to aid in diagnosing a patient. The display can be located in view of the patient. In some embodiments, the process 2100 can output the report to multiple displays including a display in view of the patient and a display located in view of the physician. The report can be included in an EMR. In some embodiments, the process 2100 can output the report and/or the raw genomic risk scores to an insurance company. The process 2100 can then proceed to 2136.

At 2136, the process 2100 can receive certain information from the medical record of the patient. The patient may have previously opted-in to a program to allow the information to be used for future refinement or retraining of one or more disease risk predictor models. The process 2100 can provide the information, which can include one or more genomic risk scores, actual patient data indicating the presence of a disease such as diabetes, the age of a patient when the one or more genomic risk scores were generated, etc. The information from the medical record of the patient may be updated over time with diagnosis codes, and used to refine one or more polygenic disease risk score predictor models. For example, over time, the process 2100 could learn that a given patient had a genomic risk score of, e.g., 50% for diabetes, but did not actually wind up with a diagnosis of diabetes based on EMR records. The process 2100 can then proceed to 2140.

At 2140, the process 2100 can provide the information from the medical record of the patient to a storage medium such as the first memory 2008. The information can be included in the genomic datasets. The process 2100 can then end.

If the requestor was a private user, then it may be desirable to provide different information in an output report. At 2144, the process 2100 can generate a user report based on one or more received polygenic disease risk scores that is suitable for a user who requested the scores him or her-self. In one embodiment the user may have logged into a web portal to provide background information and make a request, similar to the manner in which a user might request other genomic-based analysis online. When the report is ready, it can be presented to the user through the same portal. The report can include the actual polygenic disease risk scores (e.g., “raw data”), only those scores that are significantly above average, deviations from the standard risks, and/or charts, graphs, and/or other visual aids generated based on the polygenic disease risk scores. The report may also include one or more suggestions to the patient such a visual indicator suggesting to the patient that they seek a particular type of blood test, recommended interventions such as diet or exercise plans, or that they see a particular type of specialist doctor, or that they need to consider other measures like quitting smoking, etc. or links to relevant information about certain diseases. The process 2100 can then proceed to 2148.

At 2148, the process 2100 can output the report to at least one of a display and a memory for storage. The display can be a remote user interface such as the remote user interface 2024, can be located in view of the patient. The report can be included in an EMR. In some embodiments, the process 2100 can output the report and/or the raw genomic risk scores to an insurance company. The process 2100 can then end.

In another embodiment, either the user, the physician, or the server could report to an insurer or other third party certain data concerning the test. For example, impact of longevity information may be provided to a life insurer. Or, the fact that a user participated in the program may be provided to a health insurer, or to various research projects for monitoring incidence of certain diseases population-wide.

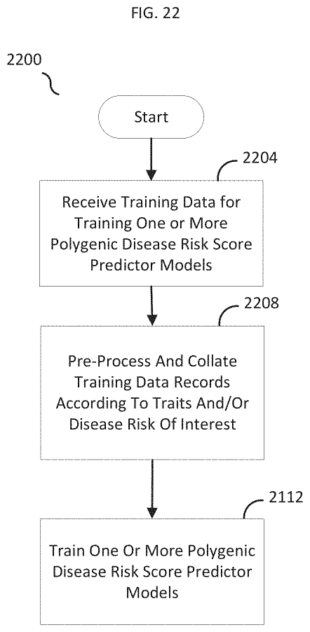

Turning now to FIG. 22, a flow chart is shown demonstrating one exemplary method for training and updating a predictor model based on genomic and other patient data. At step 2204, the process 2200 can receive training data for training one or more polygenic disease risk score predictor models. The training data can be a portion of the genomic datasets stored on the first memory 2008 of the system described above, or could be stored on a remove server. The training data can include various information associated with a number of patients. For example, for each patient record included in the training dataset, there may be stored certain categories of phenotype data, such as basic biographic data like age value, gender, a self-reported ethnicity, and the like. The records may also include more detailed phenotype information about the patients, including time series test or measurement data, such as height, weight, cholesterol levels, various hormone levels, urine analyses, and other tests. Additional data, such as parental/sibling height, weight, diagnoses, and the like may also be included. Additionally, the records may contain a number of genotype values as well as medical condition and diagnosis information (which could include ICD codes or natural language). The age value can be the age of a given patient, for example “43,” and the sex value can be the sex of the given patient, for example “male.” Each genotype value can be associated with a single-nucleotide polymorphism (SNP), and simply give the state of the SNP from a genotyping analysis that was performed for the patient. The ethnicity value can indicate a geographical region of the world the patient is most closely genetically related to, for example British. The medical condition information can include phenotype case or control data (i.e., “yes” or “no”) corresponding to whether or not the patient has and/or has had one or more conditions such as Hypothyroidism, Type 2 Diabetes, Hypertension, Resistant Hypertension, Asthma, Type 1 Diabetes, Breast Cancer, Prostate Cancer, Testicular Cancer, Glaucoma, Gout, Atrial Fibrillation, Gallstones, Heart Attack, High Cholesterol, Malignant Melanoma, and/or Basal Cell Carcinoma. The process 2200 can then proceed to 2208.

At step 2208, the process 2200 then continues to a step of “pre-processing” the training datasets. This step may first include quality-control checking each record. For example, a quality-control check may be performed for the genotypic data, to determine whether the genotype data is valid, not-corrupted, and whether the data for each record suffices for purposes of generating a predictor model. Similarly, quality control checking of the phenotype data may be conducted as well, including determining whether valid, non-corrupted data exists for non-genotype fields of the record. During this stage, the process may also determine which categories of phenotype information are available for each record, so as to determine whether the record can be used as a case or control for subsequent predictor model generation for specific traits or diseases. (E.g., if no height data is available for a record, the process will not categorize it as valid for use in generating a predictor model for height). The process may also, at this step, use natural language processing to parse narrative information in miscellaneous fields of a record, looking for terms that may be worth flagging for subsequent human review. For example, if a “Notes,” “History,” or other field of a record includes text that might be indicative of the patient having heart disease (e.g., words used like “stent” or “bypass”) the process may flag the record for a human reviewer to assess whether the record should be included as a “case” or “control” for a predictor model of various cardiovascular diseases. Alternatively, the process might auto-generate a message to the patient or the patient's healthcare provider requesting confirmation (e.g., an ICD or diagnosis code) of the possible condition.

Some data formatting may also take place during the pre-processing step. For example, measurement data may be converted to a uniform system of measurement (e.g., pounds to kilograms, or feet to meters) and various diagnostic codes (e.g., ICD9 and ICD10) may instead be replaced by common indicators. In one embodiment, multiple ICD9 or ICD10 codes, or other coding systems (e.g., non-US based codes), user self-reported diagnoses, and natural language indications may be converted into a homogenous value indicating the presence of a certain diagnosis.

In one embodiment, there may exist a set of known traits or disease conditions for which the process will generate predictor models. For each predictor model, the system may have stored an indication of the required data fields necessary for a record to qualify as a case or control for the corresponding trait or condition. For example, for a predictor of Gout, the minimum required data fields may include a set of SNPs, age, and gender. Any records that contain those fields will be tagged (e.g., an additional field added, or a lookup table entry made) as eligible for use for the given predictor. In another embodiment, a there may be an optimal and one or more sub-optimal but acceptable sets of minimum required data fields for a given trait or condition. For example, for a predictor of Type II Diabetes, the optimal set of minimum required data fields may include a set of certain SNPs, age, gender, parental diagnoses of Diabetes, and certain historical weight measurements at key ages. An alternative set of conditions may include merely those certain SNPs, age and gender, or a different set of SNPs (for example SNPs that are correlated with the optimal ones), or a subset of the SNPs. Each record that undergoes the pre-processing step could be tagged as being eligible for each of the alternative possible predictors corresponding to the alternative sets of minimum required data fields. Once the process 2200 has pre-processed the training data, the process 2200 can proceed to 2212.

At 2212, the process 2200 can train one or more polygenic disease risk score predictor models. The polygenic disease risk score predictor models can be stored on the second memory 2012. Each model can be used to predict a risk score for a specific medical condition such as Hypothyroidism for a specific ethnicity value, for example British. The process can train each model using the techniques described above in the “Model Training Algorithm” section.

Each model can include two submodels. The process 2200 can train a first submodel by regressing against the SNPs included in the medical condition data associated with each patient. For the first submodel, the process 2200 can regress against the SNPs using the LASSO technique to minimize the objective function (E.1). The process 2200 can train a second submodel by regressing the medical condition data (i.e., phenotype y=(1, 0)) against sex and age. The model can then output a single polygenic risk prediction score calculated by summing scores output by the first submodel and the second submodel. The process 2200 can then end.

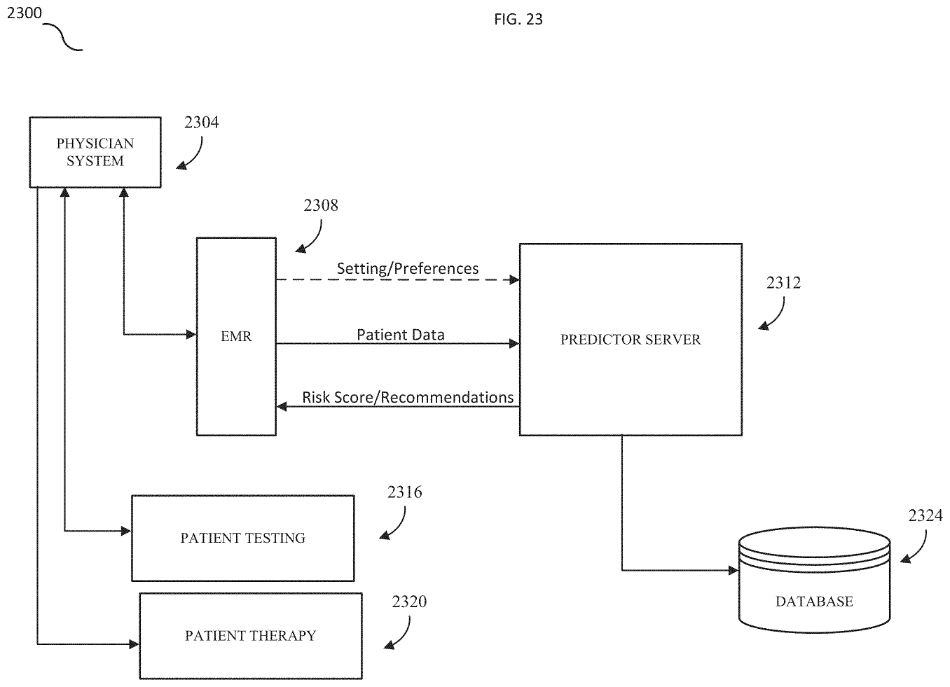

Referring now to FIG. 23, an exemplary system 2300 for predicting one or more genomic risk scores of a patient is shown. The system 2300 can include a physician system 2304 including one or more computers operated by a physician. The physician system 2304 can be in communication with a patient testing facility or lab 2316, a patient therapy facility (such as a hospital or clinic) 2320, and an electronic medical record (EMR) database 2308 having an EMR of the patient stored within. In one embodiment, the physician system 2304 may allow a physician to order a polygenic analysis for a given patient. That order may trigger several actions. First, the physician system 2304 may determine whether sufficient data exists in the patient's EMR already to fulfill the minimum data requirements of a polygenic predictor of interest. For example, if a genomic analysis already exists in the EMR, as well as existing height measurements, then that data can be used for a polygenic height assessment. If insufficient data exists, the physician system 2304 may automatically order additional testing (e.g., a urinalysis, genotyping, or various other tests) from the lab 2316. The physician system 2304 can optionally send settings and preferences to a predictor server 2312, such as settings governing which default predictors will be run against all patient records and how the results of the predictors are communicated back to the physician system and/or patient. In one embodiment, the physician system 2304 can cause the EMR database 2308 to send patient data and optionally setting and/or preferences to a predictor server 2312 in communication with the EMR database 2308. The predictor server 2312 can include one or more trained predictor models for various diseases and/or traits as described above. In one embodiment, the predictor server updates the physician system with minimum data requirements for each available type of predictor. For example, as more data is obtained by the predictor server, additional SNPs, different SNPs, or other patient phenotype data may become more important to predictions in revised and updated predictor models. The predictor server 2312 can predict genomic risk scores and/or generate recommendations for the patient based on the patient data, which can include genomic data and actual patient measurements such as height, weight, etc. The predictor server 2312 can output and generated results to a database 2324 for long term storage as well as to the EMR database 2308. The EMR database 2308 can then provide the results, which can include risk scores and/or recommendations, to the physician system 2304. The physician system 2304 may prompt the physician to decide whether to prescribe certain therapies or to undertake additional testing, based upon the risk scores and/or recommendations.

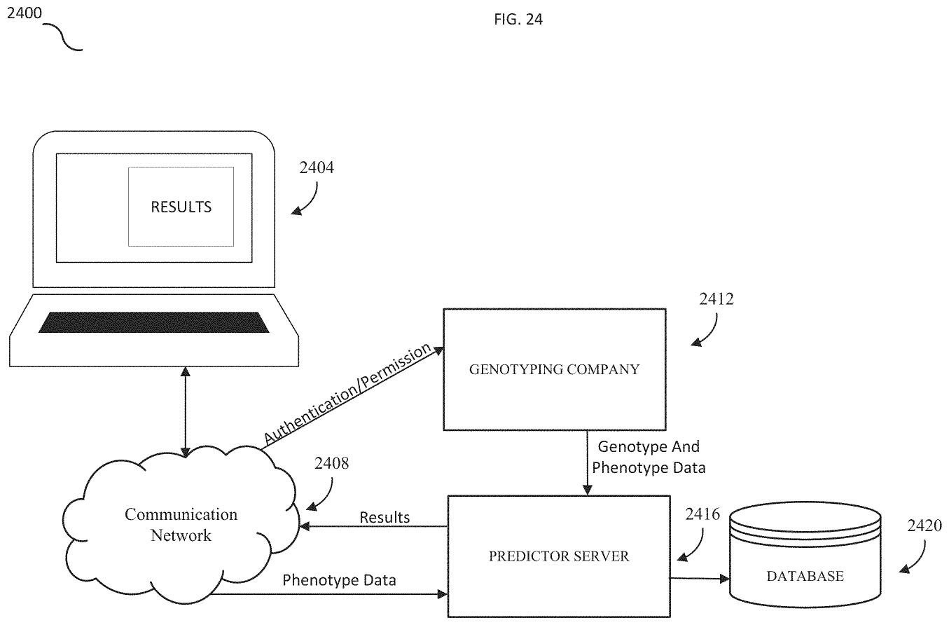

Referring now to FIG. 24, an exemplary system 2400 for providing previously generated genotype and phenotype data to a trained predictor model is shown. The system 2400 can include a patient computational device 2304 that can be a laptop computer, desktop computer, tablet computer, etc. The patient computational device 2304 can be in communication with a communication network 2408 in further communication with a predictor server 2416 and a genotyping company 2412. The predictor server 2416 can be in communication with the genotyping company 2412. Fia the patient computational device 2304, a user may log into a website of a company operating the predictor model server. That website may then open a Java applet or other window in which a user enters credentials or provides an authorization for their account with a genotyping company. Thus, the user device can request permission from and/or provide authentication credentials to the genotyping company 2412 to cause the genotyping company 2412 to provide genotype (optionally also phenotype data) directly to the predictor server 2416. The website may also ask the user to input specific phenotypic data that is necessary for the user's desired predictors. The predictor server 2416 can then generate results including one or more genomic risk scores and/or a report generated based on the genomic risk scores and provide the results to a database 2420 for long term storage and/or to the user computational device 2404 via the communication network 2408, e.g., displayed within a short time on the same webpage.

Various designs, implementations, and associated examples and evaluations of polygenic disease risk score predictor models have been disclosed. These polygenic score predictor models address the limitations of existing work by introducing more accurate risk predictor models. The present invention has been described in terms of one or more preferred embodiments, and it should be appreciated that many equivalents, alternatives, variations, and modifications, aside from those expressly stated, are possible and within the scope of the invention.

What is claimed is:

1. A method for generating a complex genomic predictor model comprising:
obtaining a set of genomic data;
pre-processing the genomic data set for at least one characteristic of interest;
computing a set of additive effects that minimize an objective function for the characteristic of interest in the pre-processed genomic data set; and
determining a polygenic risk score predictor model for the at least one characteristic of interest.

2. The method of claim 1, where in the pre-processing step includes removing records of the genomic data set that lack one or more points of information having significant variance for the characteristic of interest of the predictor model.

3. The method of claim 2 wherein the pre-processing step includes utilizing known associations between the at least one characteristic of interest and non-genomic factors are used to cull the data set.

4. The method of claim 1 further comprising regressing against single-nucleotide polymorphisms (SNPs) of the genomic data set using a modified LASSO technique to minimize the objective function.

5. The method of claim 4 further comprising regressing against phenotype data of the genomic data set, and utilizing both the SNPs regression and the phenotype regression to determine the polygenic risk score predictor model.

6. The method of claim 1 wherein the step of computing the set of additive effects includes applying a penalty term and sequentially adjusting the penalty term to allow more nonzero components in a soft-thresholding function until a Donoho-Tanner phase transition suggests a minimum number of SNPs will allow for recovery of the characteristic of interest.

7. The method of claim 1 further comprising setting aside a number of records of the genomic data set for in-sample validation of the polygenic risk score predictor.

8. A method for providing a polygenic risk score, comprising:
obtaining genotype data associated with an individual;
pre-processing the genotype data;
inputting the genotype data to a polygenic risk score predictor model, wherein the predictor model was developed through a penalized, modified LASSO regression applied to determine a set of predictor SNPs from a training genomic data set;
obtaining at least one risk score of a trait of interest for the individual from the predictor model; and
outputting a report based on a risk score for the trait of interest for the individual, according to user output preferences.

9. The method of claim 8 wherein the step of pre-processing the genotype data comprises determining whether the genotype data includes a minimum threshold of predictor SNPs of the predictor model.

10. The method of claim 9 wherein the minimum threshold includes predictor SNPs identified by the penalized regression technique used to develop the predictor model, as well as SNPs correlated with the identified SNPs.

11. The method of claim 8 wherein the user is a medical practitioner, and outputted risk score is presented in a report comparing the risk score to other risk factors associated with the trait for the individual.

12. The method of claim 8 wherein the report compares a predicted height value of the individual for the individual's age and gender, to the current height of the individual, and includes an assessment of whether the individual is on track to reach the predicted height.

13. The method of claim 8 wherein the risk score for the trait reflects a risk of the individual developing a disease condition, and the report includes recommended interventions associated with the disease condition.

14. The method of claim 8 further comprising obtaining historical medical information from the individual's electronic medical record, and wherein the predictor model comprises two submodels, one based on SNPs and one based on non-genomic medical information.

15. A system for providing polygenic risk scores, the system comprising:
a processor;
at least one memory associated with the processor, the memory comprising:
a database of training records, each record comprising genomic information of an individual and at least one characteristic of the individual;
a set of instructions which, when executed by the processor, cause the processor to:
receive genotype information for a user;
pre-process the genotype information to determine whether a threshold of SNP information is present;
provide the genotype information to a polygenic risk score predictor model;
output a report for the user based upon the result of the polygenic risk score predictor model; and
update the database of training records with the genotype information for the user, based on user consent.

16. The system of claim 15 wherein the instructions further cause the processor to update the polygenic risk score predictor model using the updated database of training records.

17. The system of claim 16 wherein the instructions further cause the processor to receive non-genomic medical information for the user, and to update the database of training records with the non-genomic medical information being associated with the genotype information for the user.

18. The system of claim 17 wherein the non-medical information includes diagnosis codes for the individual.

19. The system of claim 15 wherein the instructions further cause the processor to provide the genotype information to multiple polygenic risk score predictor models associated with multiple diseases.

20. The system of claim 19, wherein the instructions further cause the processor to:
pre-process the genotype information to determine whether a threshold of SNP information is present for each of the predictor models;
provide the genotype information only to those predictor models for which the threshold of SNP information exists; and
update those predictor models using a penalized, modified LASSO regression using the training database supplemented with the genotype information.
